(19) United States
(12) Patent Application Publication
(10) Pub. No.: US 2024/0185432 A1
(43) Pub. Date: Jun. 6, 2024

(54) SYSTEM, METHOD, AND APPARATUS FOR TRACKING A TOOL VIA A DIGITAL SURGICAL MICROSCOPE

(71) Applicant: Digital Surgery Systems, Inc., Goleta, CA (US)

(72) Inventors: George C. Polchin, Goleta, CA (US); Stephen C. Minne, Goleta, CA (US); Kyle Williams, Goleta, CA (US)

(21) Appl. No.: 18/553,955

(22) PCT Filed: Apr. 6, 2022
(86) PCT No.: PCT/US22/23650

§ 371 Date: Oct. 4, 2023

Related U.S. Application Data
(60) Provisional application No. 63/171,190, filed on Apr. 6, 2021.

(51) Int. Cl. G06T 7/20; A61B 34/20; G06V 10/25; G06V 10/44

(52) U.S. Cl. CPC G06T 7/20; A61B 34/20; G06V 10/25; G06V 10/44; A61B 2034/2065; G06T 2207/10056; G06V 2201/07

(57) ABSTRACT

The present disclosure relates generally to a system, method, and apparatus for tracking a tool via a digital surgical microscope. Cameras on the digital surgical microscope may capture a scene view of a medical procedure in real time, and present the scene view to the surgeon in a digitized video stream with minimal interference from the surgeon. The digital surgical microscope may process image data from each scene view in real time and use computer vision and machine learning models (e.g., neural networks) to detect and track one or more tools used over the course of the medical procedure in real-time. As the digital surgical microscope detects and tracks the tools, and responds accordingly, the surgeon can thus indirectly control, using the tools already in the surgeon's hands, various parameters of the digital surgical microscope, including the position and orientation of the robotic-arm-mounted digital surgical microscope.

### PRIORITY CLAIM

The present application claims priority to and the benefit of U.S. Provisional Patent Application 63/171,190, filed Apr. 6, 2021, the entirety of which is incorporated herein by reference.

### BACKGROUND

Surgeons are craftspeople who rely strongly on their hands and/or feet to achieve their objectives during surgery. During surgical and microsurgical procedures for which a microscope is used, control of the surgical microscope, particularly its position and orientation, often involves physical input from the surgeon's hands and/or feet. This reliance on the hands and/or feet can cause unnecessary interruptions in the surgery, place the surgeon off-balance in the case of foot pedal use, increase procedure time, or otherwise negatively impact patient outcomes.

Surgeon hand-based systems may include hand controls, as the hands are often the primary tools for controlling various features on existing surgical microscopes. However, hand controls may often require the surgeon to remove one or more of their hands from their primary task of controlling their surgical tools. Thus, hand controls may negatively impact surgical time and may impair the surgeon's focus and flow.

Another system for assisting surgeons involves foot controls. Legacy foot pedals may include foot-controlled buttons for performing simple functions and a joystick for motion control. Many surgeons are averse to using a foot pedal for microscope control during sensitive surgical maneuvers because of the high level of control required by their hands for controlling the surgical tools. Using the foot pedal can cause the surgeon to shift their bodyweight on the foot that is not controlling the foot pedal, and this shift can put the surgeon off-balance. Additionally, there is often a scarcity of space around the surgeon's feet owing to other devices offering foot pedals. These other devices that also offer foot pedals may remain in place for the whole procedure regardless of whether these other devices are actually in use.

Another system for assisting surgeons involves voice controls. Voice control can work well for simple commands (e.g., “increase light” or “zoom in”) that do not necessarily involve motion. However, having voice control for more extensive or motion-based commands, such as controlling the motion of a robot during surgery, may not be advised due to the greatly increased specificity required, as well as risk of patient injury.

Another system for assisting surgeons involves the use of navigation or tracker tools. For example, SYNAPTIVE'S MODUS V may provide microscopic motion and optics control functionality. However, such features are costly as they involve bulky navigation targets added to the tools. The targets may either add on to existing tools or may be built into a proprietary costly toolset that may need to be purchased by the user. Add-on targets may involve an extra registration step at procedure time to inform the system of tool geometry relative to the add-on tracker target. In such cases, the tracker target may need to be kept in view of the navigation camera, which can become onerous or may cause intermittent disruptions due to objects blocking the line of sight between the navigation camera and the tool-mounted target(s).

Various embodiments of the present disclosure address one or more of the above shortcomings.

### SUMMARY

The present disclosure relates generally to a system, method, and apparatus for tracking a tool via a digital surgical microscope. In some embodiments, cameras on the digital surgical microscope may capture a scene view of a medical procedure, and present the scene view to a surgeon in a digitized video stream with minimal interference from the surgeon. The digital surgical microscope may process image data in real time and use computer vision and machine learning models (e.g., neural networks) to detect and track one or more tools used over the course of the medical procedure in real-time. As the digital surgical microscope detects and tracks the tools, and responds accordingly, the surgeon can thus indirectly control various parameters of the digital surgical microscope, including, but not limited to, the position and orientation of the robotic-arm-mounted digital surgical microscope using the tools already in the surgeon's hands. For example, the digital surgical microscope may follow the movement of the tools and/or detect user intent based on the surgeon's use of the tools, and present relevant video data stream accordingly.

In an example, a method of tracking a surgical tool in real-time via a digital surgical microscope (DSM) is disclosed. The method may include receiving, in real-time, by a computing device having a processor, image data of a surgical video stream captured by a camera of the DSM. The surgical video stream may show a surgical tool of interest. The computing device may apply the image data to a first trained neural network model to determine a location for a bounding box around the tool of interest. For example, a first input feature vector may be generated, based on the image data, and may be applied to the first trained neural network model to generate a first output feature vector. The first output feature vector indicates the location for the bounding box around the tool of interest. The method may further include generating an augmented image data comprising a bounding box around the tool of interest. The augmented image data may be applied to a second trained neural network model to determine a distal end point of the tool of interest. For example, a second input feature vector, based on the augmented image data, may be generated and applied to the second trained neural network model to generate a second output feature vector. The second output feature vector may indicate the location for the distal end of the tool of interest. The method may further include causing, in real-time by the computing device, the DSM camera to track the distal end of the tool of interest.

In some embodiments, the applying the image data, the generating the augmented image data, the applying the augmented image data, and the causing the DSM camera to track the distal end of the tool of interest may be responsive to an identified displacement. For example, the computing device may identify, based on a previously received image data of the surgical video stream, a displacement of a feature in the surgical video stream beyond a threshold distance. Furthermore, the computing device may cause the DSM camera to track the distal end of the tool of interest by adjusting a field of view of the DSM camera such that a focus point associated with the distal end of the tool of interest is at the center of the field of view. In some aspects, the focus point may be at a predetermined distance from the distal end of the tool of interest in a direction towards the displacement.

Another example method of tracking a tool in real time via a digital surgical microscope is disclosed. The method may include receiving, by a computing device having a processor, and from one or more image sensors associated with the digital surgical microscope, image data of a first segment of a medical procedure. The one or more image sensors may be focused towards a first position of a surgical area of the medical procedure. The method may further include applying, by the processor, a neural network to the image data to detect a tool (e.g., used in the medical procedure). The method may further include tracking, by the processor, movement of the tool through one or more subsequent segments after the first segment. After detecting a displacement of the tool beyond a threshold distance, the processor may cause the image sensors to be refocused towards a second position of the surgical area of the medical procedure.

In an example, a system for tracking a surgical tool in real-time via a digital surgical microscope (DSM) is disclosed. The system may comprise a DSM comprising a DSM camera, a processor, and a memory device. The memory device may store computer-executable instructions that, when executed by the processor, causes the processor to perform one or more methods or steps of a methods described herein. For example, the instructions, when executed, may cause the processor to: receive, in real-time from the DSM, image data of a surgical video stream captured by the DSM camera, wherein the surgical video stream shows a tool of interest; apply the image data to a first trained neural network model to determine a location for a bounding box around the tool of interest; generate an augmented image data comprising a bounding box around the tool of interest; apply the augmented image data to a second trained neural network model to determine a distal end point of the tool of interest; and cause, in real-time, the DSM camera to track the distal end of the tool of interest.

Additional features and advantages of the disclosed method and apparatus are described in, and will be apparent from, the following Detailed Description and the Figures. The features and advantages described herein are not all-inclusive and, in particular, many additional features and advantages will be apparent to one of ordinary skill in the art in view of the figures and description. Moreover, it should be noted that the language used in the specification has been principally selected for readability and instructional purposes, and not to limit the scope of the inventive subject matter.

### BRIEF DESCRIPTION OF THE FIGURES

FIG. 1 is an illustration of markings on a surgical tool, according to an example embodiment of the present disclosure.

FIG. 2 is a perspective view of a trackable target, according to an example embodiment of the present disclosure.

FIG. 3 shows a diagram of an example deep learning model used for tracking a tool via a digital surgical microscope, according to an example embodiment of the present disclosure.

FIG. 4 is a perspective view of an image tacking a tool, and provided by a digital surgical microscope, according to another example embodiment of the present disclosure.

FIG. 5 shows a flow diagram of a method of training a deep learning model for generating a bounding box around a tool of interest, according to an example embodiment of the present disclosure.

FIG. 6 shows a flow diagram of a method of training a deep learning model for detecting a distal end of a tool of interest from image data, according to an example embodiment of the present disclosure.

FIG. 7 shows a flow diagram of a method of applying deep learning models for detecting and tracking a tool of interest from image data in real-time, according to an example embodiment of the present disclosure.

FIG. 8 is an illustration showing an application of a semantic segmentation model, in accordance with a non-limiting embodiment of the present disclosure.

FIG. 9 is a diagram showing a method of tracking a tool, via a digital surgical microscope, according to an example embodiment of the present disclosure.

FIG. 10 shows an example system for tracking a tool via a digital surgical microscope, according to an example embodiment of the present disclosure.

FIGS. 11A-11I show illustrations of example tools of interest, according to example embodiments of the present disclosure.

FIG. 12 shows an illustration of an example non-relevant tool, according to example embodiments of the present disclosure.

### DETAILED DESCRIPTION OF EXAMPLE EMBODIMENTS

The present disclosure relates generally to a digital surgical microscope that can provide visualization during many types of medical procedures. Tools used in the medical procedures may be prominently visible to the digital surgical microscope, as the digital surgical microscope can capture the view of the scene in a digitized video stream. In some aspects, computer vision techniques (e.g., including standard image processing as well as machine learning) may be used to facilitate the detection, tracking and recording of the tools, including the pose of the tools relative to the microscope, over the course of the medical procedure in real time. Such detection and tracking can allow the surgeon to have better control of the microscope and its parameters, including, but not limited, to the position and orientation of the robotic-arm-mounted digital surgical microscope using the tools already in the surgeon's hands. Therefore, the surgeon can keep their hands occupied with their primary task of performing surgery with tools, instead of pulling out to adjust the microscope. This may enhance patient care by reducing unnecessary interruptions during surgery, thereby reducing the overall procedure time and improving surgeon's focus.

In detecting and tracking the tools, the stereoscopic camera of the digital surgical microscope can become a localizer for the tools relative to the microscope, akin to a localizer in a surgical navigation system. Tool pose relative to patient anatomy can also be determined via one or more methods, including, but not limited to: using surgical navigation tools, including, but not limited to a separate localizer device to determine relative poses of patient anatomy and microscope camera; performing patient surface extraction using the digital surgical microscope as an extended localizer to locate a multitude of surface points on the patient anatomy; or a combination thereof. Such knowledge of tool pose relative to patient anatomy may be stored and analyzed for use in next-tool and next-process-step prediction, post-market surveillance, surgeon training, and case review.

One or all of the cameras composing or other multi-view camera of the digital surgical microscope maybe used for the detection, identification and tracking of surgical tools and other items typically seen in the view of the digital surgical microscope. Various embodiments of the present disclosure describe systems and methods for identifying uniquely tools used during the procedure, and can detect and track salient point(s) on the instrument relative to tracked targets. For example, combinations of novel and nonobvious computer vision and machine learning techniques, tool marking, lighting, and optical filtering schemes are described for unique identifying the tools used during a procedure.

In some aspects, the presently disclosed digital surgical microscope may provide a touchless microscope control. For example, the digital surgical microscope may allow the user to use, as the controller, the tracked tool in the scene and/or multiple tracked tools in the scene in combination.

In some aspects, the presently disclosed digital surgical microscope may facilitate post-market surveillance, e.g., of tools used in surgical procedures. Thus, the digital surgical microscope may facilitate an important required step for medical device development & production. For example, the digital surgical microscope can uniquely identify each tool used during a procedure and can allow the recording of tool usage, the sequence of tool usage, and/or timing of tool usage.

With navigation capabilities, the digital surgical microscope may provide tracking, recording, and the concurrent and subsequent display of tool paths relative to patient anatomy over the course of the medical procedure. The tracked and displayed tool paths may accurately be within a tolerance level of the actual paths taken by the tools.

In some aspects, systems and methods presented herein may display information to the user of the digital surgical microscope about tools currently being used in an ongoing medical procedure. The information may include, for example, a name of a tool, a function of the tool, a running time of the use of the tool, a total running time for the tool, a manufacturer of the tool, a model number of the tool, a serial number of the tool, etc. Some of these information may be available as part of a universal device identifier (UDI).

In some aspects, systems and methods presented herein can predict any subsequent tools that may be required or expected in an ongoing medical procedure. The prediction may help medical personnel (e.g., the scrub nurse) reduce time in selecting and retrieving tools (e.g., to be readily accessible by the surgeon) as the medical procedure proceeds further. In some aspects, a checklist of tools which have been used since the start of a medical procedure, and/or are still expected to be used, may be displayed (e.g., on a user interface presented by the digital surgical microscope).

In some aspects, a machine learning model (e.g., a neural network) may be trained on the appearance of tools used in medical procedures. The training data for the machine learning model may rely on features received from an imaging system comprising the digital surgical microscope and in the context of the specific and various surgical procedures in which the respective tools are used. The trained machine learning model may be used to detect and track unmarked tools with no other added targets.

The features used in the trained machine learning model may be classified, broadly, as “marked tools” and “markerless tools” or “unmarked tools” as will be described herein. Tradeoffs exist between classifying tools in these categories. Marking the tool may be a deterministic way to identify a tool in a straightforward manner but may involve preparing or otherwise having the knowledge of the actual tools to be used. The markerless technique (classifying a tool as a “markerless tool” or an “unmarked tool”) may not necessarily require preparation of the actual instance of the tool used, and may instead depend on one or more machine learning networks (variously referred to as: deep learning; deep learning models; deep learning networks), which may require a priori information about the appearance of the tool.

### Detecting and Tracking Marked Tools

Several methods may be used to mark a given tool directly and indirectly. Many surgical tools are manufactured from stainless steel. Laser etching can provides a means of marking such tools directly. Other direct marking methods may involve adding retroreflectors. Indirect means of marking tools may include adding a marked sleeve to the tool. The marked sleeve may be sterile and biocompatible. Furthermore, the marked sleeve may be heat-shrinkable or non-heat-shrinkable. Therefore, the sleeve may include, but is not limited to, an add-on (e.g., a single-use and/or sterilizable) marked structure; a medical grade printed heat shrink tubing which may be made to fit tightly, e.g., by initial assembly not to be heated necessarily; or a medical grade printed heat shrink tubing that may be sterilized multiple times and can also be printed with USP Class VI inks. Also or alternatively, the sleeve may include an attached locator cord for later accounting and removal from surgical site. Various forms of tool markings are thus described herein.

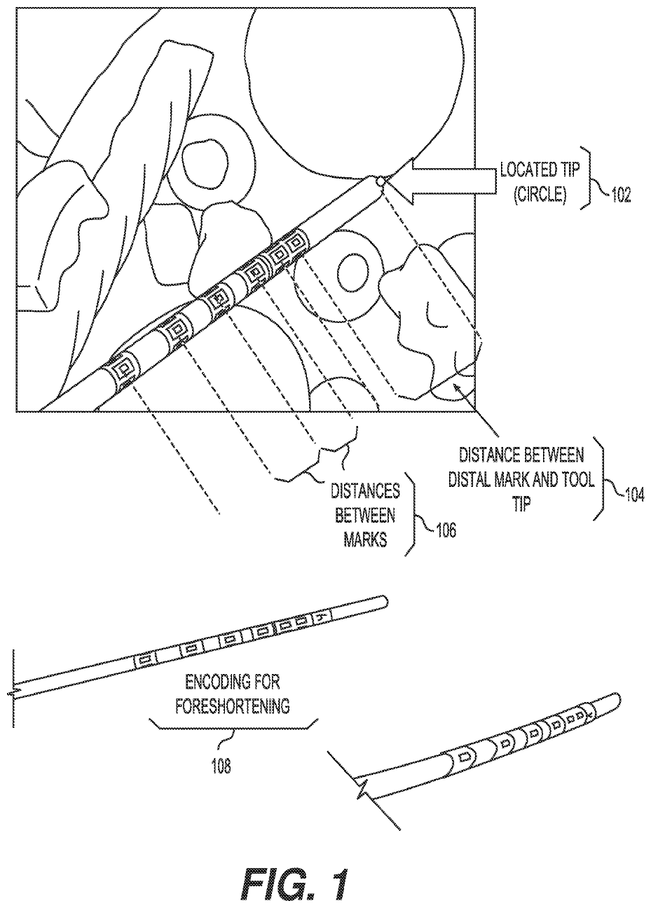

At least one example of marking tools involves axial marks or marking based on ratios of ring distances across the cylindrical axis of the tool. Many surgical tools (e.g., suction cannulas) may comprise at least one cylindrical-style shaft. An example system and method of marking along the axis of the cylindrical-style shaft is thus described. A series of rings may be marked on the shaft. Each ring may proceed partially or completely around the shaft. The set of rings may be distributed along the shaft such that the ratios of distances between rings may be known. Also or alternatively, the ratios between adjacent pairs of rings may be unique and/or predetermined. To add robustness, distances between non-adjacent pairs of rings may be calculated and added to the set of expected ratios. The distance to the tooltip from the most distal mark may be known by design or by a registration step. The ratios between such rings may be preserved over a wide range of angles regardless of the angle between the tool and the camera. Thus, marking a tool based on predetermined and/or unique identifiable ratios of distances between rings may be an excellent choice for medical procedures involving the digital surgical microscope. In at least one example of the present disclosure, markings on the tools may be encoded for foreshortening, as will be shown by way of FIG. 1.

FIG. 1 is an illustration of markings on a surgical tool, according to an example embodiment. As shown in FIG. 1, tools may often be oriented to be reaching down into a channel or opening in the surgical site, and therefore may proceed away from the camera. For example, the FIG. 1 shows a tool tip 102 (e.g., located by the system) that is somewhat buried downwards into a surgical site. Thus, any marks on the tool shaft may appear to be foreshortened to the camera. For example, the distances between marks 106, and the distance between the distal mark and the tool top 104 on the tool shown in FIG. 1 shows an example of foreshortening (e.g., through the seemingly decreasing distance between marks). The above described “ring distance ratio” method of marking may be robust to such foreshortening. Reference 108 shows an example of an encoding for foreshortening. Other methods and forms of markings, such as a “hole-in-shape” marking, may be stretched along the axis distance to compensate for foreshortening.

In some aspects, information may be encoded into the marks. For example, clever use of marking spaces, size, shape, and the like, information may be encoded into the marks. Such information may be used for determining tool identity and the poses of salient tool points relative to the marks. Error correction similar to QR code error correction can also be built into the encoded marks, e.g., when enough bits of data are able to be encoded. For challenging environments, such as a thin tool shaft (e.g. 1 mm or less in diameter), simpler encodings may be necessary, which may often reduce the ability to encode error correction. However, even in such cases, the marks may be robust to missing or false positive marks through the use of distance ratios for example.

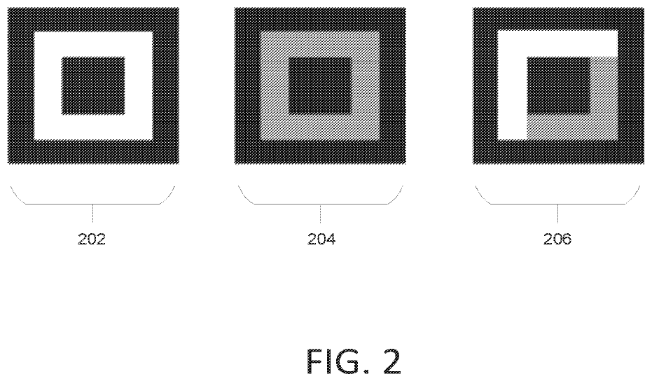

FIG. 2 is a perspective view of an image sensor, according to another example embodiment. The image sensors used by the digital surgical microscope (e.g., digital surgical microscope camera) may be robust to fluid presence. As shown in FIG. 2, the image sensor(s) of the digital surgical microscope may be nominally standard RGB CMOS color sensors such that the use of color in the system can enhance performance. For example, the use of the red channel in such a sensor can enable the detection of the standard QR location pattern on the left image 202 even as it is fully or partially occluded with red blood as shown in the center image 204 and right image 206, respectively. In the red channel, the center image 204 and the right image 206 may resemble the left image 202, at the expense of some resolution.

In some aspects, tool markings may be based on a Universal Device Identifier (UDI) marking requirement for surgical tools, which may be a directive from health authorities to provide deterministic identification for medical equipment. In further aspects, tool markings may include laser-engraved quick response (QR) codes, which can provide not only a detectable and trackable pattern but also encode a variable amount of information. Where such codes are useable, the QR code may be used to encode relevant device information, for example, an index into a list of tools that may be stored elsewhere (e.g., a memory module of the digital surgical microscope. The use of multiple such targets about the tool can make their use much more robust to occlusion and difficult imaging conditions. There may be certain device types for which QR code use is challenging, for example on rounded surfaces with a small radius (e.g. radius <1.5 mm). Thus, other methods and forms of marking are described herein.

In at least one aspect of the present disclosure, radially asymmetric markings may be used on tools. For example, for tools with a shaft which have a bend or an angle to it, as some suction cannulas do, radially symmetric markings may be insufficient to determine tip location. However, markings that vary angularly about the axis may be used in such devices. Also or alternatively, a registration step may be used in such instances.

Systems and methods presented herein for tracking tools via the digital surgical microscope may also account for tool modification or alteration during the medical procedure. For example, some tools (e.g., suction cannulas) may be designed to be reformable during use to allow access to dynamically changing approach paths. Such changes in tool shape may be detected in the case of marked tools by comparing the predicted tip location to actual scene data from the camera(s). A registration step may be suggested to the user (e.g., via a message on a user interface of the digital surgical microscope) when such change in tool shape has been detected. In the case of unmarked tools (e.g., where a tool tip is found dynamically), such changes in tool shape may be automatically detected and/or registered.

Markings on the tools, and their detection by the digital surgical microscope, may be robust to medical operations where the tools are not optimally visible. For example, the depth of field of the digital surgical microscope is limited which means that parts of any given scene might not be optimally focused. The extended depth of field of the digital surgical microscope compared to an optical microscope mitigates this complication. Additionally the markings are fairly robust to non-optimally focused images.

The tracking of tools via the digital surgical microscope may be robust to medical operations occurring over a wide range of magnification. The varying magnification (e.g., zoom size) of the digital surgical microscope can allow smaller targets to be used at higher magnification. However the range of magnification used in a given procedure may dictate that a range of target sizes be used. Also or alternatively, scale-invariant targets (e.g., such as axial rings) may be used instead, or in addition to, the QR-type codes.

The tracking of tools via the digital surgical microscope may also utilize other types of tool markings, including, but not limited to, hole-in-shape, STAG, April tags, and ARuco markings. A hole-in-shape is a method of providing a dark region completely surrounded by a light shape, such as the “finder” marks in a QR code. Standard image processing techniques (e.g., such as contour finding) can allow the digital surgical microscope to track the location of such shapes by finding contours, which may have a set number of contours (e.g., only one contour) inside of the shapes. Arranging the marks cleverly as described further herein can facilitate the encoding of data, such as orientation and tool identification. The contrast in shading (e.g., light regions within dark shapes) can also or alternatively be used in this method of marking tools.

In some aspects, the digital surgical microscope may provide coaxial lighting and/or utilize retroreflectors. In such aspects, retroreflective markings may be used in some embodiments to mark and identify tools. The retroreflective markings are arranged in a pattern or patterns on the tool such that these patterns are easily visible to the camera during normal tool use and uniquely identify tools to a desired degree of individual identification of tools and/or tool types. For example all 3 millimeter suction cannulas might have the same pattern, but a more advanced tool might be identifiable down to the individual instance of such a tool. In some aspects, the retroreflective patterns may be realized using retroreflective glass beads.

Thus, the color, spacing and/or pattern of individual marks and groups of marks may be rendered to uniquely identify each tool in a given group of tools in use for a given medical procedure. When detected, the color, spacing and/or pattern may be parsed (e.g., by an image processor associated with the digital surgical microscope). The parsing may lead to a direct information (e.g. as QR codes provide) of the tool. Also or alternatively, the parsing may lead to an index which can then be used to look up into a map of tools for extended tool information.

To increase the market range of the product, a service may be offered to mark the customer's existing tools to enable the tools to work within the system. This can include laser marking and/or the use of “marked sleeves.”

Systems and methods presented herein for tracking tools via the digital surgical microscope may be able to handle varying target visibility during the medical procedure. The systems and methods may utilize a number of features to allow it to handle varying target visibility. For example, the systems and methods may utilize marker redundancy to make the system robust to partial occlusion. Furthermore, multiple targets may be distributed about the tool, e.g., to enable the robustness to orientation as well as orientation determination. Even further, the systems and methods may leverage multi-scale targets, e.g., to enable operation over varying degrees of magnification.

In some aspects, tool points may be registered to the tool markings. For example, when a single mark is used, e.g., on a simple axial tool with no moving parts (e.g., as in a suction cannula), the registration may be described as “The Real and Virtual Surgical Marking Pen”. Registration can typically occur at some time before the surgical procedure, for example, at design time or marking time, often far before the surgical procedure.

Generally, tools may be registered by design. The markings may be placed during manufacturing with sufficient pose accuracy relative to the salient points of the tool. This can allow the use of a canonical registration for such tools without the need for a separate registration at some time again before the use of the tool. For tools that are marked a posteriori, that is, existing tools that are upgraded to work with the system, a separate registration step may be performed.

Machine Learning Models to Detect and Track Tools of Interest that May be Unmarked

As will be discussed, systems and methods are presented herein for tracking markerless tools. To avoid the need to mark tools, a markerless technique is implemented, as will be discussed. The markerless technique may rely on one or more machine learning networks which may be variously referred to as: artificial neural networks; deep learning; deep learning models; deep learning networks; deep learning neural networks; deep neural network (DNN). In one embodiment, a specialized version of a DNN, e.g., a convolutional neural network (CNN) may be used, especially as CNN is suited for image processing.

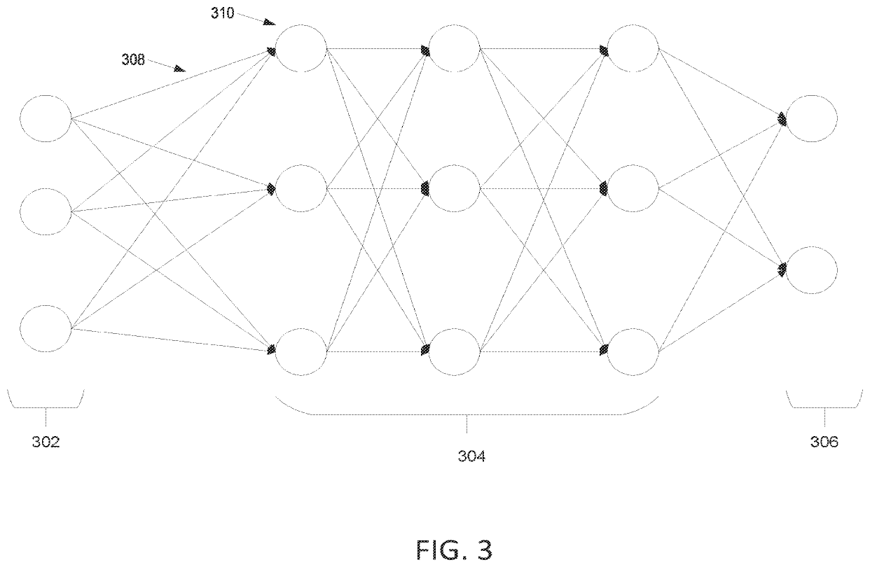

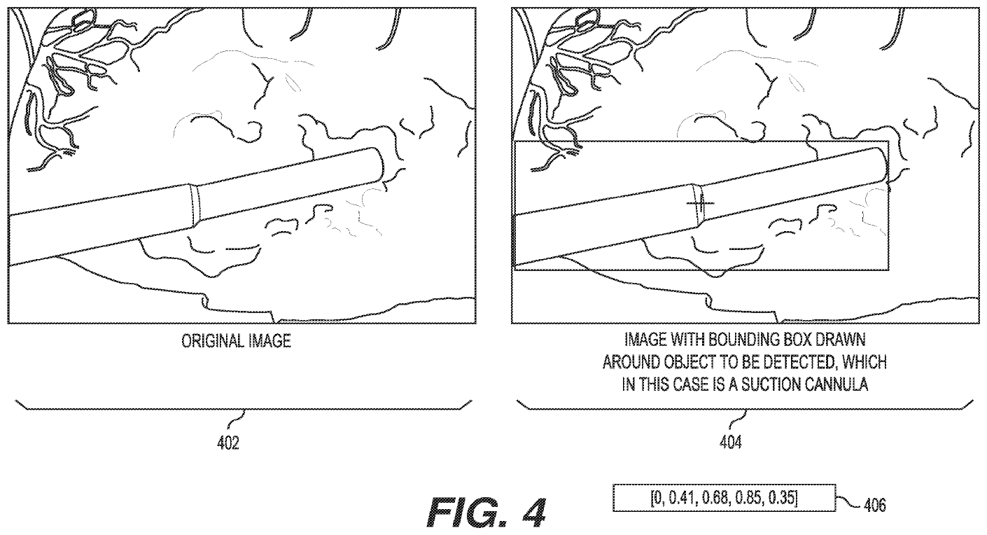

FIG. 3 shows a diagram of an example deep learning model used for tracking a tool via a digital surgical microscope, according to an example embodiment of the present disclosure. As shown in FIG. 3, a node 310, referred to in DNN as a neuron 310, may be a basic building block of a DNN. One or more neurons may be grouped into a layer (e.g., layers 302, 304, and 306). A deep learning network can typically have an input layer 302, one or more hidden layers 304, and an output layer 306. Within each layer, there may be one or more connected neurons, and neurons may connect across layers. Typically, most neurons may be connected to many other neurons. Inputs and outputs of neurons (e.g., the network as a whole) may be represented numerically. The neurons may implement a set of mathematical functions on the numeric input(s) to create one or more numeric outputs. The mathematical functions may include, for example, a multiplication by some weight(s) 308, an addition of some bias(es), and an application of some typically nonlinear processing via an activation function. A node containing a combination of the above described functions may be referred to as a perceptron. The mathematical function (e.g., activation function) may depend on a node from one layer to a node in the next layer, and may rely on a weight 308 assigned between each nodes. The weights 308 may be initialized and may be subsequently adjusted through an iterative processes (e.g., backpropagation). The weights, biases and other configuration values used in the network may be determined during a training phase. In some embodiments, the neural network or networks may be trained via supervised learning. Supervised learning may take as an input a (typically very large) set of labeled information. A labeled information may comprise information where a human processes an input, for example, an image from the digital surgical microscope, and writes out a label in the form of a computer-readable file comprising numeric information. An example of labeled information is shown in FIG. 4.

FIG. 4 is a perspective view of an image tacking a tool, and provided by a digital surgical microscope, according to another example embodiment. FIG. 4 shows an original image 402 and a labeled image 404. A label may be associated to an image in which the label may include information on whether a certain class of surgical instrument (e.g., a suction cannula) is visible in the image, and where that instrument is located in the image. For example, a human might draw a bounding box around the relevant part of the image containing the object as shown in image 404 of FIG. 4. The numeric output for such a bounding box is represented numerically by values for each of several features. For example, five numbers that may represent the bounding box may comprise: a class of an object (e.g. the number 0 may be assign to correspond to “suction cannula”); a location of the center of the box along a Cartesian (e.g., x and y) axis, relative to a pre-established coordinate system of the image (for example with the origin at the upper left of the image and the positive X axis proceeding to the right of the image and the positive Y axis proceeding toward the bottom of the image; and a width and a height of the bounding box. To make the labeling robust, the location with respect to the X-axis and width numbers may be normalized to the width of the image. Furthermore, the location with respect to the Y-axis and the height may be normalized to the height of the image. Thus an example label might include an array or vector of values such as “0, 0.41, 0.68, 0.85, 0.35” shown by marker 406.

In some embodiments, the input to the neural network may be an image from the digital surgical microscope. To speed training and development as well as to increase runtime performance, the stereoscopic image of the digital surgical microscope may be reduced to a monoscopic image (e.g., using a “left-eye image) and may be reduced in resolution. For example, the width and the height of the image can each by a value (e.g., 4) to produce a lower resolution image (e.g., from a 1920×1080 pixel image to a 480×270 pixel image.) In the training phase for generating the neural network, an activation function may be chosen. Starting weights and biases may be assigned, and a plurality of labeled images may be fed through the network. Each image input can result in an output from the network. The output can then be compared to the numeric information in the label corresponding to the input. The resulting set of outputs may be compared to the expected outputs as given in the labels. Based on the comparison, the weights, biases, and other associated parameters in the neural network may be adjusted to reduce the error or loss. The above described process may be iterated numerous times (e.g. tens of thousands of times) and the resultant set of weights, biases, and any other related parameters (possibly millions of individual numbers) constitute the trained network model. In some aspects, other neural networks, machine learning models, and methodologies may be utilized equally effectively in this invention.

In some embodiments, a neural network or machine learning model may be pre-trained, and/or imported, e.g., from an external server. In some aspects, a pre-trained network may be used and the transfer learning method may applied. The pre-trained network may comprise a trained network model which may have been developed by experts and trained to recognize a somewhat large plurality of everyday objects. Transfer learning may comprise a method of re-training a trained network model to recognize classes of objects not present in the original training set of said model. The output layer(s) and possibly some small amount of hidden layers may be retrained using labeled training images of these specific object classes. The amount of such training images for transfer learning may be smaller than that required for training the full network from scratch. Thus, transfer learning can help alleviate labor-intensive tasks like labeling, which may involve human input.

In some aspects, the training of the machine learning model (e.g., neural network) can happens offline, e.g., before runtime. The trained model may be output as a model architecture along with weights, biases, and/or other configuration parameters developed by the training phase. As used herein, an inference may refer to a runtime use of the trained model with its configuration parameters in place. The input may be put into the input layer of the model. The output or outputs can thus be inferred from that input. As used herein, a prediction may also refer to the output of the model. A probability may often accompany the output(s), and may describe the likelihood that the detected object and other output parameters are actually correct.

Computation during runtime may be performed via processor modules connected informationally to, or located inside, the digital surgical microscope. The processor modules may be referred to herein as inference modules and may operate on a variety of hardware, including, but not limited to: a generic central processing unit (CPU) processor (e.g., INTEL CORE I7-9700K); a generic graphics processing unit (GPU) (e.g., NVIDIA GTX 1070); a specialized GPU tailored to perform inference (e.g., a “tensor core” present in NVIDIA'S VOLTA and TURING architectures); a dedicated inference device (e.g., INTEL® NEURAL COMPUTE STICK 2)′ or cloud-based solutions on the Internet (e.g., AMAZON WEB SERVICES).

As many images of the video stream as possible, single image frames captured by the digital surgical microscope may be sent to the inference module for processing. To improve performance, the stereoscopic image in some embodiments may be pruned to just one view, for example, to just the left eye. Also or alternatively, the stereoscopic image may be reduced in resolution. For example, the stereoscopic image may be scaled down in its width and its height, e.g., so that it is ¼ the original width and ¼ the original height, e.g., using the OpenCV computer vision function, cv::resize( ).

The processing rate of the inference module might not be able to keep up with the frame generation rate of the camera in which case some incoming frames are ignored. In other embodiments, the reduction in resolution may be performed but the original resolution of the stereoscopic image may also be maintained or used (e.g., after being scaled down separate from each other). For example, such methods may allow triangulation to be used to determine tool location in some desirable space, such as patient data space as described herein.

In various embodiments, the stereoscopic image may be used as a single image, and inference can occur on it as a whole, returning, for example, two results (e.g., one result for each view present in the image) when a tool is detected. In various embodiments, the resolution may be kept at the native resolution of the digital surgical microscope (e.g., 1920×1080 per eye for a total of 3840×1080).

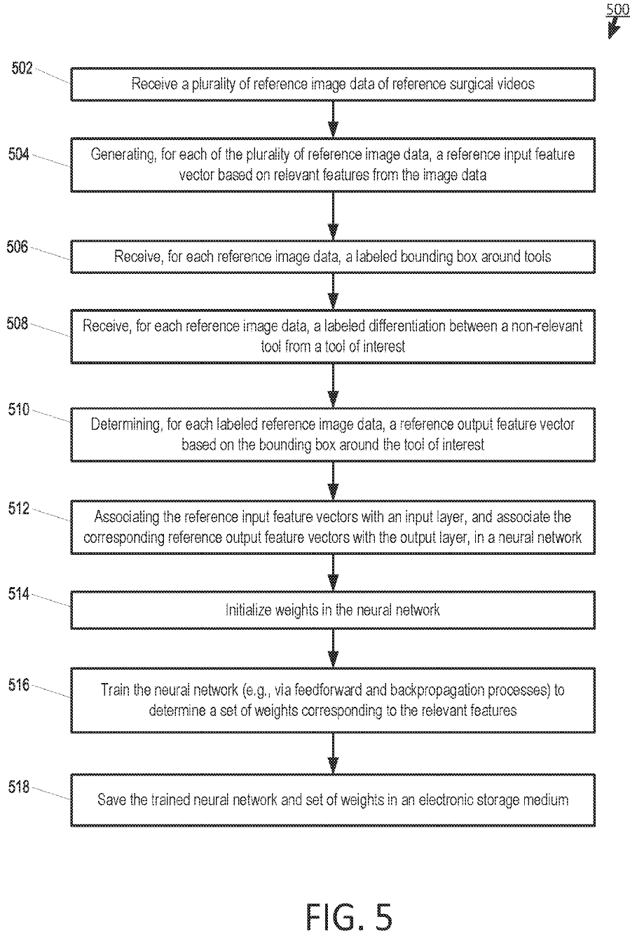

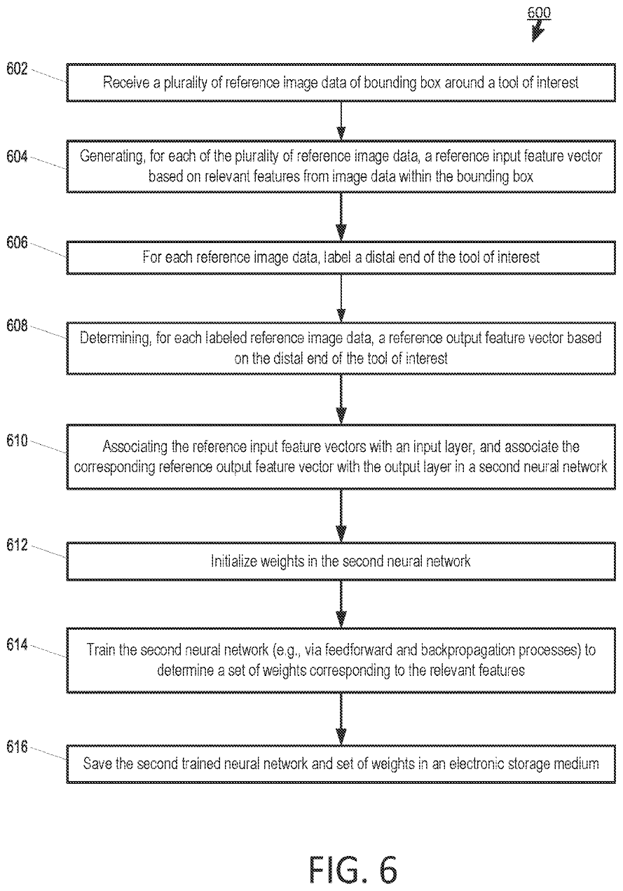

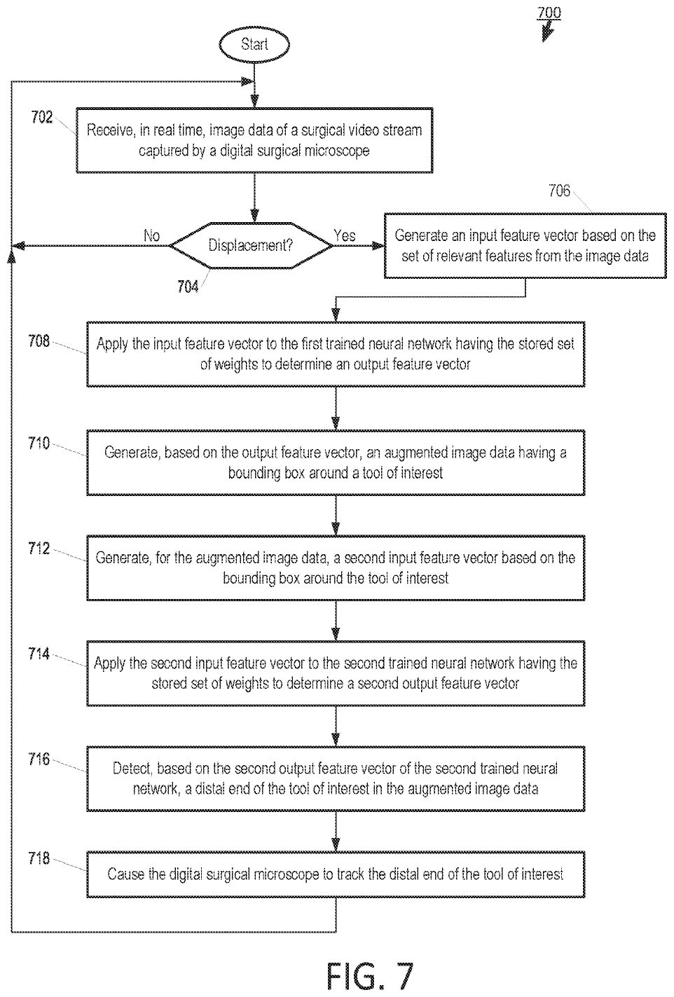

Neural networks may be used in various modes. For example, as will be described in FIGS. 5 and 7, a first stage neural network may be trained and applied to generate a bounding box around a tool of interest. A heuristic may be used to determine the distal end of a tool of interest (e.g. suction cannula). Furthermore, as will be described in FIGS. 6 and 7, a second stage deep learning, and/or a differently-trained first stage neural network may be used to determine distal ends of a tool of interest more deterministically per monoscopic image, e.g., by using training images with the distal tip bounding box labeled. A stereoscopic (or other multi-camera, also known as multi-view) determination of bounding box and distal end can allow the determination of distal end locations in a coordinate space of a camera. In some aspects, further calibration and registration can allow the transformation to other desirable space, e.g., microscope space and patient data space.

FIG. 5 shows a flow diagram of a method 500 of training a deep learning model for generating a bounding box around a tool of interest, according to an example embodiment of the present disclosure. One or more steps of method 500 may be performed by a computing device having a processor. For example, the digital surgical microscope may comprise or may be associated with a computing device. Processor modules may be connected informationally to, or located inside, the digital surgical microscope. The processor may execute one or more steps or methods discussed herein (e.g., in FIGS. 5-7) based on computer-executable instructions stored in a memory device of the computing device.

Method 500 may begin with the computing device receiving a plurality of reference image data of reference surgical videos (step 502). As used herein, an image data or a surgical video may be a reference image data or a reference surgical video to distinguish from the image data or the surgical video being received in real-time in in which a tool is detected and tracked by the digital surgical microscope. For example, reference image data from reference surgical videos, and reference feature vectors may be used for the training of machine learning models to apply to image data being received in real-time.

In some embodiments, the reference image data and/or the reference surgical videos may be obtained from the surgical site, surgery room, or medical facility used for surgery in which the trained machine learning models are to be eventually applied, in real-time, for the automated tracking of tools of interest (e.g., in method 700 of FIG. 7). Also or alternatively, the reference image data and/or the reference surgical videos may be obtained from images or surgical videos of the surgeon operating and/or the surgeon's tool employed during the surgery in which the trained machine learning models are to be eventually applied, in real-time, for the automated tracking of tools of interest (e.g., in method 700 of FIG. 7). By training neural network models using training data directly obtained from images, settings, and/or tools on which the trained neural networks are to be applied, the neural network models may be more specific, and therefore more effective and accurate for their end goals (e.g., the first neural network model for determining a bounding box around a tool of interest (e.g., as explained in FIG. 5), and the second neural network model for detecting a distal end of the tool of interest (e.g., as will be explained in FIG. 6)).

At step 504, the computing device may generate, for each of the plurality of reference image data, an input feature vector based on relevant features from the image data. For example, relevant features may include, but are not limited to, the color of an individual pixel of the image data, a contrast of an individual pixel in relation to neighboring pixels, a brightness of an individual pixel, a degree of similarity with a neighboring pixel, a curvature. In some embodiments, for example, where a convolutional neural network model is to be implemented, the feature vectors may be automatically determined based on the convolved image data. In such embodiments, the relevant features may not necessarily be features that a human could relate to.

As previously discussed, a supervised machine learning model may involve learning from training data, e.g., a set of image data comprising both input feature vector (e.g., the image data) and an output feature (e.g., a label). Supervised learning may involve processing an input, for example, an image from the digital surgical microscope, and writing out a label in the form of a computer-readable file comprising numeric information. For example, each reference image data may be labeled (e.g., manually) by generating a bounding box around the surgical tools shown in each image frame of the reference surgical video stream. The surgical tools of each reference image data may further be labeled as a non-relevant tool (e.g., a respirator) or a tool of interest. In one embodiments, a non-relevant tool may comprise a suction tool (e.g., a Frazier suction), whereas tools of interest may include other (e.g., non-suction) tools. For example, tools of interest may include, but are not limited to: a scapel, a knife, forceps (e.g., a tissue forceps, a babcock forceps, a bipolar forceps (Bovie), an Oschner forceps, etc.), a needle, a retractor (e.g., a spinal self retaining retractor, a handheld retractor, a Senn retractor, a Gelpi retractor, a Weitlaner retractor, etc.), rongeur (e.g., a Kerrison Rongeurs, a Pituitary Rongeur, a Leksel Rongeur, etc.), a hemostats trocar, a Yankauer, a knife handle with or without a blade, a bonnet, a straight and curved hemostats, an iris scissor, a Pennfield freer, a ball tip probe, a nerve hook, a curet, a posterior cobb elevator, an osteotome, etc. The computing device may thus receive, for each reference image data, a labeled bounding box around tools (block 506) and a labeled differentiation of tools (e.g., a non-relevant tool versus a tool of interest) (block 508). The labelling may allow the computing device to recognize and differentiate tools that include suction tools (e.g., non-relevant tools) and non-suction tools (e.g., tools of interest). In one aspect, the first network model or another nested neural network model may be trained and detect all tools from reference image data while a subsequent neural network model (e.g., the first neural network model discussed in method 500) may be trained and used to detect tools of interest.

At block 510, the computing device may determine, for each labeled reference image data, an output vector based on these labels. For example, the output vector for the bounding box and the tool differentiation may be represented by values for each of several features. In some embodiments, five numbers that may represent the bounding box may comprise: a class of an object (e.g. the number 0 may be assign to correspond to “suction cannula”); a location of the center of the box along a Cartesian (e.g., x and y) axis, relative to a pre-established coordinate system of the image (for example with the origin at the upper left of the image and the positive X axis proceeding to the right of the image and the positive Y axis proceeding toward the bottom of the image; and a width and a height of the bounding box. To make the labeling robust, the location with respect to the X-axis and width numbers may be normalized to the width of the image. Furthermore, the location with respect to the Y-axis and the height may be normalized to the height of the image. Thus an example label might include an array or vector of values such as “0, 0.41, 0.68, 0.85, 0.35” shown by marker 406.

At block 512, the computing device may associate the input feature vectors with an input layer of a neural network model, and may associate the corresponding output vector with the output layer in the neural network model. For example, in the neural network model shown in FIG. 3, the computing device may input values from the reference feature vectors of block 504 into the nodes of input layer 302. The computing device may input values from the output vector thus formed in block 510 on to the output later 306. The neural network model thus formed, referred to herein as first neural network model, may be distinguishable from a second neural network model formed in method 600 (e.g., shown in FIG. 6), as will be discussed.

At block 514, the computing device may initialize weights in the first neural network model. In some aspects, biases may be initialized and provided for any layer of the first neural network model. The weights and biases may affect an activation function occurring at a node. As will be described, the weights and/or biases may be adjusted based on iterative processes in the training of the first neural network model.

At block 516, the first neural network model may be trained to determine a set of weights corresponding to the relevant features. A first neural network model may be deemed trained, for example, if the model may accurately predict (e.g., to within a threshold level) a location for a bounding box around a tool of interest within an image data. A first neural network model may be trained, for example, if it yields an optimized set of weights for use in the application of the trained neural network. The optimized set of weights may be determined through iterative feedforward and backpropagation processes, e.g., as previously described in FIG. 3. As will be described in method 700 (e.g., in FIG. 7), the trained neural network model may be subsequently applied to image data of a live surgical video stream, for the computing device to accurately generate a bounding box around a tool of interest. The trained neural network may then be saved (e.g., to cloud and/or an electronic storage medium) for use in identifying, and/or generating the bounding box around, the tool of interest from the live surgical video stream (block 518).

The bounding box may comprise a rectangle or other quadrilateral. Thus, an output vector for the bounding box may include, for example, aspect ratios for the rectangle. Also or alternatively, the output vector for the bounding box may include the length, width, and/or a vertex angle of the quadrilateral, and/or a location of a vertex of the bounding box within the image (e.g., by coordinate). In some embodiments, the bounding box may comprise a square, a circle, or other curved shape. Such embodiments may obviate the need to determine aspect ratios (e.g., due to the length and width of a square or a circle being the same), and may thus speed up the training process.

FIG. 6 shows a flow diagram of a method of training a deep learning model for detecting a distal end of a tool of interest from image data, according to an example embodiment of the present disclosure. One or more steps of method 600 may be performed by a computing device having a processor. Moreover, the deep learning model trained in method 600 (e.g., referred to herein as a second neural network model or second neural network) may differ from the neural network model trained in method 500, as the second neural network model may be used to detect a distal end of a tool of interest from image data, after the first neural network model has been used to identify the tool of interest (e.g., via a bounding box).

Method 600 may begin with the computing device receiving a plurality of reference image data comprising a bounding box around a tool of interest (block 602). The plurality of reference image data may be obtained from reference surgical videos. In some embodiments, the plurality of reference image data may be those reference image data received in method 500 and already labeled with the bounding box around the tool of interest. For each of the plurality of reference image data, the computing device may generate a reference input feature vector based on relevant features from image data within the bounding box (block 604). Moreover the image data within the bounding box may correspond to the tool of interest. For example, relevant features may include, but are not limited to, the color of an individual pixel of the image data, a contrast of an individual pixel in relation to neighboring pixels, a brightness of an individual pixel, a degree of similarity with a neighboring pixel, a curvature. In some embodiments, for example, where a convolutional neural network model is to be implemented, the feature vectors may be automatically determined based on the convolved image data. In such embodiments, the relevant features may not necessarily be features that a human could relate to.

In some embodiments, the reference image data may be obtained from the surgical site, surgery room, or medical facility used for surgery in which the trained machine learning model is to be eventually applied, in real-time, for the automated tracking of tools of interest (e.g., in method 700 of FIG. 7). Also or alternatively, the reference image data may be obtained from images or surgical videos of the surgeon operating and/or the surgeon's tool employed during the surgery in which the trained machine learning model is to be eventually applied, in real-time, for the automated tracking of tools of interest (e.g., in method 700 of FIG. 7). By training neural network models using training data directly obtained from images, settings, and/or tools on which the trained neural networks are to be applied, the neural network models may be more specific, and therefore more effective and accurate for detecting the distal end of the tool of interest.

In order to train the second neural network model to detect a distal end of a tool of interest (e.g., when image data is fed into the model), each reference image data may be labeled so as to indicate the distal end of the tool of interest. The labeling may be done manually and then stored in the computing device as training data. The computing device may thus receive this labeled reference image data (block 606). In some aspects, the computing device may receive the plurality of reference image data earlier at block 602 as already having the labeled distal end of the tool of interest. The computing device may determine, for each labeled image data, a reference output feature vector based on the distal end of the tool of interest (block 608). In some embodiments, the output feature vector may comprise a set of values indicating a location, within the bounding box, of the distal end of the tool. In some aspects, the set of values may further include a direction of (e.g., trajectory) of movement of the distal end of the tool.

At block 610, the computing device may associate the input feature vectors with an input layer, and associate the corresponding reference output feature vector with the output later in a second neural network. For example, in the neural network model shown in FIG. 3, the computing device may input values from the reference input feature vectors of block 604 into the nodes of input layer 302. The computing device may input values from the reference output feature vector thus formed in block 610 on to the output later 306. Moreover, the resulting neural network (second neural network) is different from the neural network formed in method 500, as the purpose, and the underlying training data used for that purpose, is different. Whereas the training data in method 500 includes reference input feature vectors associated with relevant features of reference image data mapped to reference output feature vectors associated with the bounding box around a tool of interest, the training data in method 600 includes reference input feature vectors associated with image data within a bounding box around a tool of interest mapped to reference output feature vectors associated with a distal end of the tool of interest.

At block 612, the computing device may initialize weights in the second neural network. In some aspects, biases may be initialized and provided for any layer of the neural network. The weights and biases may affect an activation function occurring at a node. As will be described, the weights and/or biases may be adjusted based on iterative processes in the training of the neural network model.

At block 614, the computing device may train the second neural network model to determine a set of weights corresponding to the relevant features. The second neural network model may be deemed trained, for example, if the model may accurately predict (e.g., to within a threshold level) a location for a distal end of a tool of interest from image data of a bounding box around the tool of interest. Furthermore, the second neural network may be trained, for example, if it yields an optimized set of weights for use in the application of the second trained neural network to determine a distal end point in a tool of interest shown in a live surgical video stream. The optimized set of weights may be determined through iterative feedforward and backpropagation processes, e.g., as previously described in FIG. 3. As will be described in method 700 (e.g., in FIG. 7), the second trained neural network model may be subsequently applied to image data of a live surgical video stream, for the computing device to accurately identify a distal end of a tool of interest to allow the digital surgical camera to track it. The second trained neural network may then be saved (e.g., to cloud and/or an electronic storage medium) for use in identifying, and/or tracking the distal end of the tool of interest from the live surgical video stream (block 616).

FIG. 7 shows a flow diagram of a method of applying deep learning models for detecting and tracking a tool of interest from image data in real-time, according to an example embodiment of the present disclosure. One or more steps of method 700 may be performed by a computing device having a processor. For example, the digital surgical microscope may comprise or may be associated with the computing device. In some embodiments, the computing device performing the training of the neural network models (e.g., training of the first and second neural network models as illustrated in FIGS. 5 and 6, respectively) may also apply the trained neural network models in method 700. Alternatively, the training of one or more of the neural network models may be performed by another computing device, or may be performed externally.

Method 700 may begin with the computing device receiving, in real-time, image data of a surgical video stream captured by a digital surgical microscope (block 702). For example, as a surgeon begins a surgical procedure, the digital surgical microscope may begin recoding the surgical site. The field of view of the surgical site may include one or more surgical tools, including a tool of interest. Of the one or more surgical tools, the tool of interest may refer to a surgical tool that the surgeon is most frequently using, and/or the tool that is within the surgeon's control for the longest time, such that the surgeon can control the digital surgical microscope based on movements of the tool. The recording may generate a surgical video steam comprising a plurality of image data (e.g., a plurality of image frames in formats readable by the computing device). Each image data (e.g., image frame) may be presented to the computing device in real-time. The computing device may determine if there is a displacement (e.g., of a tool or other feature in the field of view) based on the received image data (block 704). For example, the computing device may compare a received image data, and a previously received image data, and determine whether the image data fails to satisfy a similarity threshold, such that a failure may indicate a displacement. The computing device may continue to receive image data of the surgical video stream until there is a displacement.

If the computing device finds a displacement, the computing device may generate an input feature vector based on the set of relevant features from the image data (block 706). For example, relevant features may include, but are not limited to, the color of an individual pixel of the image data, a contrast of an individual pixel in relation to neighboring pixels, a brightness of an individual pixel, a degree of similarity with a neighboring pixel, a curvature. In some embodiments, for example, where the input feature vector is to be applied to a trained convolutional neural network model, the specific feature may be automatically determined based on the convolved image data. In such embodiments, the relevant features may not necessarily be features that a human could relate to.

At block 708, the computing device may apply the input feature vector to the first trained neural network to determine an output feature vector. For example, the first trained neural network (e.g., stored at block 518 of method 500) may be retrieved from an electronic storage medium (e.g., of the computing device or an external system that trained the first neural network model). The first trained neural network model may also include a set of weights and/or biases optimized over the training process to be able to allow the neural network model to effectively determine the location of a bounding box around a tool of interest from image data. As previously discussed, a supervised neural network model undergoes training based on a training data comprising known reference output vectors for known reference input feature vectors, as was illustrated via methods 500 and 600. After the neural network model is trained, the trained neural network may be used to determine an unknown output feature vector using a known input feature vector. Thus, the input feature vector thus formed in block 706 may be applied to the first trained neural network model to determine an output feature vector. As the first trained neural network model was trained to identify a bounding box around a tool of interest from image data, the output feature vector may indicate a location for a bounding box around a tool of interest (e.g., using a set of values formed by the output feature vector).

At block 710, the computing device may generate, based on the output feature vector (e.g., thus generated from the first trained neural network model), an augmented image data having a bounding box around the tool of interest. For example, the location of the bounding box may be determined from a set of values represented by the output feature vector determined in block 708. For example, the set of values may include: a class of an object (e.g. the number 0 may be assign to correspond to “suction cannula”); a location of the center of the box along a Cartesian (e.g., x and y) axis, relative to a pre-established coordinate system of the image (for example with the origin at the upper left of the image and the positive X axis proceeding to the right of the image and the positive Y axis proceeding toward the bottom of the image; and a width and a height of the bounding box. Since the bounding box is around a tool of interest, the class of the object may be designated to be indicative of being a tool of interest (e.g., 0 being a tool of interest (e.g., the suction cannula) and 1 being a non-relevant tool). After the bounding box overlaid on the image data around a tool of interest depicted in the image data, the resulting image data formed may comprise the augmented image data. In some embodiments, a tool of interest may include but is not limited to, for example: a scapel, a knife, forceps (e.g., a tissue forceps, a babcock forceps, a bipolar forceps (Bovie), an Oschner forceps, etc.), a needle, a retractor (e.g., a spinal self retaining retractor, a handheld retractor, a Senn retractor, a Gelpi retractor, a Weitlaner retractor, etc.), rongeur (e.g., a Kerrison Rongeurs, a Pituitary Rongeur, a Leksel Rongeur, etc.), a hemostats trocar, a Yankauer, a knife handle with or without a blade, a bonnet, a straight and curved hemostats, an iris scissor, a Pennfield freer, a ball tip probe, a nerve hook, a curet, a posterior cobb elevator, an osteotome, etc. In some aspects, each bounding box may comprise a label (e.g., a value) indicating the specific tool of interest within the bounding box.

From the augmented image data, the computing device may generate a second input feature vector based on the image data comprising (or comprised within) the bounding box around the tool of interest (block 712). In some aspects, the second input feature vector may be based on relevant features of image data inside the bounding box (e.g., including the tool of interest). The relevant features may include, but are not limited to, the color of an individual pixel of the image data inside the bounding box, a contrast of an individual pixel in relation to neighboring pixels inside the bounding box, a brightness of an individual pixel inside the bounding box, a degree of similarity with a neighboring pixel inside the bounding box, a curvature inside the bounding box, etc. In some embodiments, for example, where the second input feature vector is to be applied to a trained convolutional neural network model (e.g., if the second trained neural network model is a convolutional neural network), the specific feature may be automatically determined based on the convolved image data. In such embodiments, the relevant features may not necessarily be features that a human could relate to.

At block 714, the second input feature vector may be applied to the second trained neural network model to determine the second output feature vector. For example, the second trained neural network (e.g., stored as explained in block 616 of method 600) may be retrieved from an electronic storage medium (e.g., of the computing device or an external system that trained the first neural network model). The second trained neural network model may also include a set of weights and/or biases optimized over the training process to be able to allow the second neural network model to effectively determine a distal end point of a tool of interest from image data. In some aspects, the second output feature vector may comprise a set of values indicating a location (e.g., Cartesian coordinates) of the distal end of the tool of interest in the augmented image data (and/or in the image data received at block 702). The computing device may thus detect the distal end of the tool of interest in the augmented image data (block 716).

At bock 718, the computing device may cause the digital surgical microscope to track the distal end of the tool of interest. For example, the digital surgical microscope may be moved so that the detected distal end of the tool of interest is at the center of the field of view of the digital surgical microscope. Also or alternatively, the computing device may cause the digital surgical microscope to track a point that is near to, or speculated to be contacted by, the distal end of the tool of interest. For example, the point may be at a predetermined distance from the detected distal end of the tool of interest in a direction towards a patient anatomy. The predetermined distance may be between 2 pixels and 50 pixels, for example, to the right or upper-right of a detected distal end of the tool. Tracking such a point may allow the digital surgical microscope to predict a surgeon's next area of focus, and may cause the digital surgical microscope camera to be directed there. Thereafter, the computing device may continue to receive image data of the surgical video stream (block 702), and may continue to monitor if there are any displacements (block 704), e.g., to cause the digital surgical microscope to redirect its focus, using the methods discussed.

In some embodiments, the computing device may bypass the process to determine a bounding box. For example, a neural network model may be trained on the basis of reference image data received in block 502 of method 500 from reference surgical videos. The reference image data may be labeled to indicate a location of the distal end of the tool. Input vectors based on the reference image data may be associated with output vectors based on the labeled distal end in a neural network. The trained neural network model may then be applied to the image data received in real-time at block 702 to detect the distal end of the tool of interest. Thus, such embodiments may bypass the need to apply two different neural network models (e.g., nested neural models where a first neural network model determines a bounding box and the second neural network model detects the distal end), and instead relies on a single neural network model.

In some embodiments, the computing device may rely on a single neural network model that determines a bounding box around the tool of interest, where the bounding box also indicates the distal end of the tool of interest. For example, in embodiments where the bounding box is in the form of a square, the side(s) of the bounding box that are closest to the distal of a tool may be identified. The identification of those sides may be a part of the training data. For example, reference output vectors may not only indicate the location of a bounding box but may also indicate (e.g., by values in the output vector), the sides of the bounding box that intersect at the distal end of the tool of interest. In such embodiments, the neural network model (e.g., a modified version of the first neural network model applied at block 708) may be used to directly detect the distal end of the tool of interest, thus bypassing the need to apply the second neural network model.

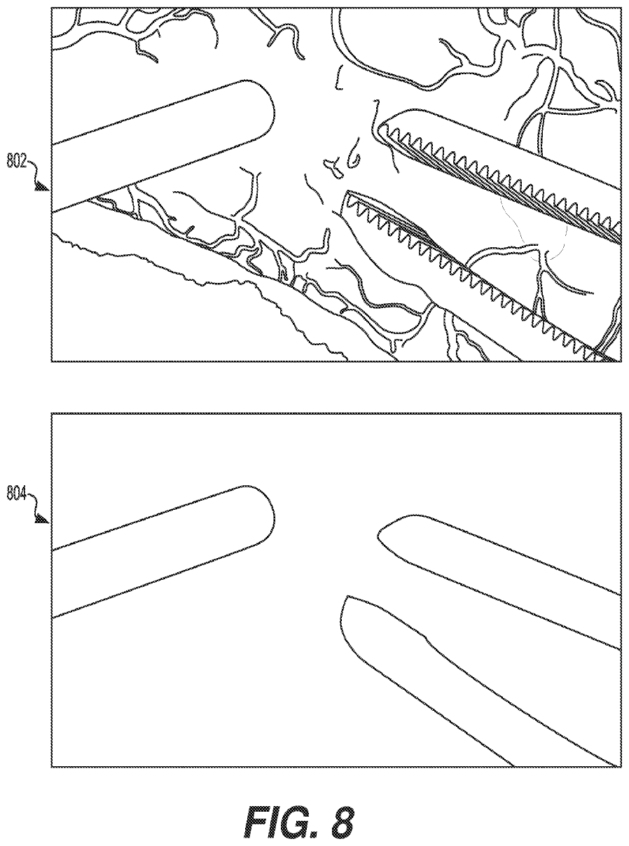

In some embodiments, a semantic segmentation model may be trained to recognize “tool” and “not tool” pixels. The computing device may then use heuristics and/or user input to determine the tool of interest and the distal end of the tool of interest. FIG. 8 is an illustration showing an application of a semantic segmentation model, in accordance with a non-limiting embodiment of the present disclosure. Specifically, FIG. 8 shows an example input 802 and output 804 for semantic segmentation. The output 804 can also representative of what training data looks like (e.g., reference image data comprising the input 802 and labeled with the output 804). The training may use computer vision techniques, such as contour determination with subsequent contour centerline determination, as well as morphological skeletonization.

In some embodiments, the location of individual tool components (e.g. tool tip) of the tool may be recorded and processed over time to discern user intent. Furthermore, the information gleaned by the detection, recognition and tracking as described herein may be recorded and/or stored over a length of time in a storage buffer in the information processor or nearby. The storage buffer may be of a finite length with data stored on a rotating pointer basis and discarded (typically overwritten) on a first-in-first-out basis.

A set of machine learning models may process the recorded series over varying lengths, e.g., to detect gestures pre-defined by the system. The detected gestures may be interpreted by the processor as user intent and converted to microscope control commands. For example, to enter and leave a given movement mode (e.g., an angular movement mode), the machine learning model or program performed by the processor may be trained to recognize a user holding the tool distal end in a given quadrant of the screen (e.g., the northeast quadrant) for a predefined amount of time (e.g., one second), moving the tool to the opposite quadrant (e.g., the southwest quadrant) over the course of same or another predefined amount of time, and then holding the tool in that opposite quadrant (e.g., the southwest quadrant) for yet some other predefined amount of time. Also or alternatively, the user may be trained to perform the same. Such movements may be substantially dissimilar to a large majority of the movements that are made by the surgeon with the tool over the course of typical procedures. Also or alternatively, controls and training regimen may be implemented to prevent the user from compromising patient safety in any way during the movement of tools for such gestures.

In some aspects, other parts of tools besides their distal end may be detected, recognized, and tracked recorded in the manners described herein, e.g., for the primary use of the tool distal end. Also or alternatively, other object types (e.g., gloved finger, gauze, suture thread, etc.) may be detected, recognized, tracked, and used in such manners.

Furthermore, cameras (also called “views”) other than the main microscope view camera may be substituted for, or used in addition to the main microscope view camera, for capturing imagery of the objects of interest. For example, one or more additional cameras mounted to the underside of the microscope head and pointing toward the scene may be a viable option. Such cameras may be implemented with different parameters from those of the digital surgical microscope. In some aspects, parameters used by the additional cameras may be better tuned to perform tasks described herein, while not being as constrained to the task of surgical site visualization as are the camera of the digital surgical microscope.

### Controlling the Digital Surgical Microscope Using the Tool Tip

Knowledge of the tooltip location in a monoscopic image or in a 3D space may be converted into microscope controls in various ways. Moving the microscope may be associated with robotic controls. The intent of controlling the microscope position can allow the surgeon to keep the region of interest of the surgical site optimally visible onscreen (e.g., nominally centered onscreen). As the medical procedure progresses, the surgical site can move for example, upward onscreen (toward “north”), e.g., so that the site and the surgeon's tools working on the site are near or beyond the field of view of the microscope. Conventionally, in such cases, the surgeon would have to pause the surgery and remove one or both hands from the site to reposition the microscope. By allowing the system to automatically detect the tooltip, the system can keep the microscope positioned such that the tooltip does not move off screen (e.g., off the field of view).

In some aspects, the system may designate a no-move zone (e.g. central region) for the microscope camera. For usability, a heuristic is used to keep the system from moving the microscope too much, thereby distracting and/or annoying the user. One or more regions are defined in the heuristic. While a set of microscope movement may be allowed in one region, a different set of microscope movement may be allowed in another region. For example, when the tooltip is found to be within a central region onscreen, no microscope movement may occur. However, when the tooltip is found to move out of that central region, the heuristic may cause the control algorithm to move the microscope such that the tooltip, in its new location, is designated as the center of a new central region. Optionally, the microscope may move into a smaller central region such that the microscope is then centered on the tooltip. FIG. 8 describes an example of the above described aspect of the present disclosure.

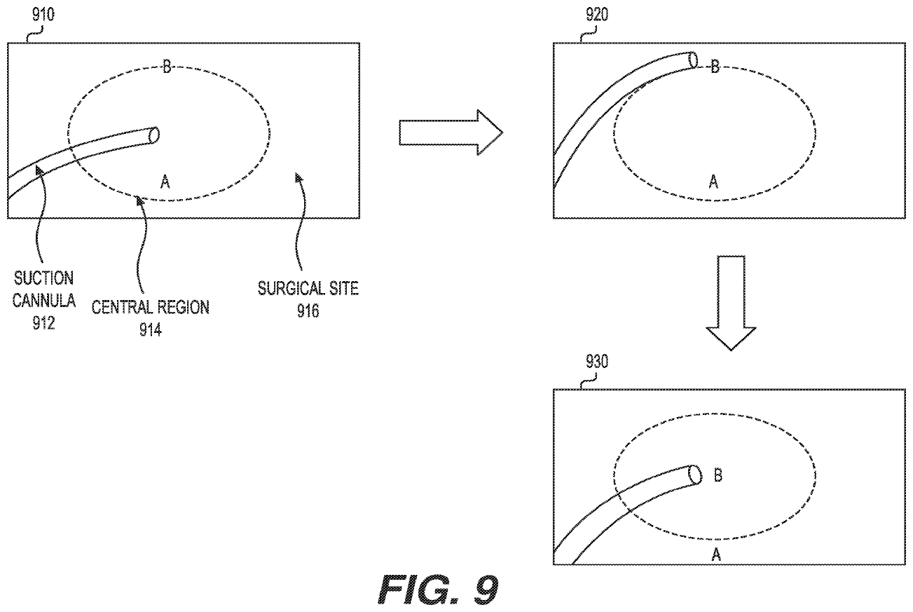

FIG. 9 is a diagram showing a method of tracking a tool, via a digital surgical microscope, according to another example embodiment. Specifically, FIG. 9 shows an example of centering the field of view of a microscope camera. For example, box 910 a segment of a medical procedure where a suction cannula 912 in the surgical site 916 is within a central region 914 (e.g., the field of view of the microscope camera). Box 920 illustrates another segment of the medical procedure, where a surgeon is moving or has moved the suction cannula “northward” or upward in the image, thereby moving the suction cannula out of the central region to location B. The deep learning network detection of the bounding box and distal end of the suction cannula are not shown explicitly. As described previously, deep learning, via a neural network, may detect that the distal end has moved out of the central region. Based on this detection, the system may activate a robotic arm movement such that the microscope view may move to bring the new suction cannula location B back into the central region (e.g., the field of view of the microscope camera), as shown in box 930.

In some embodiments, the tooltip may proceed off screen, thereby rendering it un-detectable by the detection algorithm. The user may move the microscope to follow the tooltip thereby bringing the tooltip and surgical site back into view of the microscope. The tracking algorithm may be able to detect and learn the intent of a user to bring the tool top and surgical site back into the field of view. The user may be trained to use the tooltip in such a situation to play a sort of “follow the leader” game and cause the microscope to move toward the desired position. This training may be more effectively achieved by bringing the tooltip back into view temporarily and moving it slowly enough toward the desired site that the control algorithms can keep up.

Positional movements (e.g., of the tools and/or microscope camera) may include movements along an X or Y axis onscreen, in addition to other movement regimes (e.g., roll, pitch, yaw, angular movements, etc.), as described herein. For example, detecting the orientation of the tool can allow the use of such orientation to control the orientation of the microscope.

In some embodiments, the system may facilitate user activation of movement, e.g., by discerning user intent. It may be undesirable to continually convert tool activity to certain commands. While the intent of the user to move the microscope with X-Y movement may be discerned by setting a “central region” as described elsewhere in this document, other movement controls may often be desired by the user even within the central region. Such desired movement controls may include movement controls for angular movement. To simplify detection of user intent, a starting orientation may be defined. For example, a starting orientation may be defined as when a tool is first introduced by the user and detected by the system, or when a special “orientation control” mode is entered under user control for example via a voice command or a simple handheld button connected informationally to the system. The mode may be exited in a manner similar to how it is entered. Another means of entering and/or exiting the mode is via gestures converted to user intent, as described herein.

Onscreen orientation changes of the tool may be translated to microscope movements, e.g., based on the focal point of the microscope and relative to the onscreen view. For example, if the user, after initiation, changes the tool orientation such that its angle increases about the screen's X-axis, the controller can move the robot and/or the microscope head, thus the onscreen view, such that the view about the microscope's focal point may increase its angle about the screen's X-axis. Control may be implemented similarly for the other axes.

In one embodiment the microscope control system may be set to auto-focus after some or all mechanical movements thereby keeping the autofocus region in focus continually. In another user-initiated mode, the tooltip may be used to control the focal point of the microscope. This may implemented in under user preference. For example, one method may involve an optical focus wherein the focus optics are changed to place the focus at the tooltip. Another method may involve a Z-axis mechanical movement by the robot of the microscope head such that the microscope head is kept a constant distance (the focus distance) away from the tooltip.

In some embodiments, a calibration of the camera can allow a determination of tooltip location in a known space. Calibrating the stereoscopic camera of the digital surgical microscope can provide an accurate determination of the location of features of viewed objects in camera space. The tooltip may be an example of such a feature and its position in camera space may be found at every frame where the screen position of the tooltip is determinable in each of the eyes of the stereoscopic camera of the digital surgical microscope or in each of the eyes of a multi-view camera (e.g., where a multi-view camera has any number of “eyes” as compared to a stereoscopic camera which has typically has two eyes). The calibration information can then be used to perform triangulation, and can thus allow a determination of the location of the tooltip in camera space. Prior or further calibration of the camera space to the robot space may allow a calculation of the tooltip location in camera space relative to its location. Further calibration steps may link camera space to other desirable spaces, e.g., patient anatomy space. Such knowledge can allow the use of a detected and tracked tools to be used as a pointer in the linked space to aid in surgical navigation. Such use can also provide calibration and registration integrity by having the system show the tool in the live view as well as in the patient anatomy space. The process of calibration and registration integration can point to any misalignments between or among such views, including any degree of misalignment. The degree of the misalignment may represent a degree of mis-calibration and/or mis-registration of the surgical navigation system.

The main microscope controls used most by the surgeon may be grouped into mechanical controls as described above, and optical controls (e.g., zoom capabilities). Each type of control can accept user input via gesture-to-user-intent method as described. As a complement or alternative to gesture input, generic parameter control may be used through screen centric activation of graphical menus onscreen. A tool component (e.g., the tool tip) may be used as a sort of computer mouse with control ability and adapted to not need any buttons.

One or more areas of the display of the digital surgical microscope system may be dedicated to an onscreen graphical user interface (GUI). Such areas may be placed at the edge(s) of the display as those areas may typically be outside the region of interest of the surgical site. This may be user-friendly because the tool may be detected, recognized and tracked to keep it within a central region onscreen. In some aspects, the GUI may be hidden from view. When the user directs the tool tip (or whichever object or tool part is being used for this purpose) into a GUI region, the GUI can then be shown to the user. The GUI may be divided into distinct regions of a size large enough to easily hold the tool tip inside of it for a given amount of time (e.g., one second) and the region boundaries may be shown as part of the GUI.

For example, the left edge of the display may be divided into eight equally sized squares. The squares' width and height may be, for example, one sixteenth of the width and height of the display. The squares' boundaries may be displayed when the tool tip is positioned in any of the squares for a prescribed time (e.g., 0.5 second or longer). The square nearest the upper edge of the display may be designated before runtime to represent a “zoom in” command and the square nearest the lower edge may be designated to represent a “zoom out” command. Other squares may be designated for other functions. A graphical icon describing the function of a given square may be displayed inside each square to prompt the user of the nature of the function. When the tool tip is held by the user in the “zoom in” square for more than a prescribed time for example one second, the zoom in command may be activated on the microscope either for a set amount of magnification increase or alternatively until the user directs the tool tip outside of the square. The behavior of other squares may be similar.

The GUI areas may be laid out with areas representing the range of functions desired to be controlled by the controlling object (e.g., the tool tip.) In some embodiments, the right edge of the image may be rendered to duplicate the functionality of the left edge. Also or alternatively, the right edge may contain functions that may be different from the left edge. Similar design choices may be made for the top and bottom edges. In some cases, no GUI may be present in some areas.

In some instances, the user might not be able to move the tool to the dedicated GUI area. For example, the dedicated GUI area might be physically blocked or it may not be safe to do so. In such instances, backup control options may exist for some or every control such that the user may have an alternative means to complete the desired microscope control action. Furthermore, various controls may be reachable via the microscope handles and/or via the system touchscreen input display.

### System and Network Environment for Tracking the Tool Via the Digital Surgical Microscope

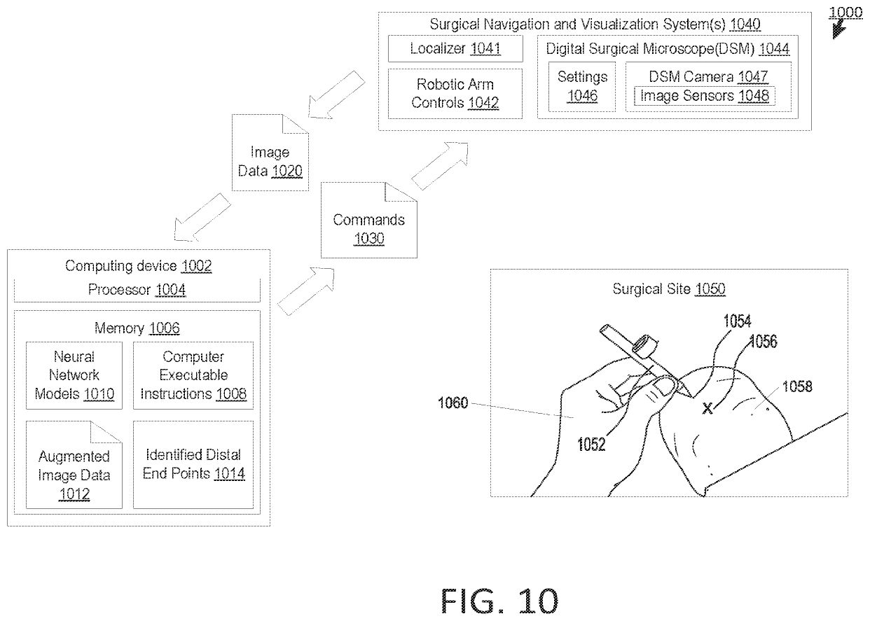

FIG. 10 shows an example system 1000 for tracking a tool via a digital surgical microscope, according to an example embodiment of the present disclosure. The example system 1000 may include a computing device 1002, surgical navigation and visualization system(s) 940, and a surgical site 1050.

The surgical navigation and visualization system(s) 1040, may include a surgical navigation system (e.g., a localizer 1041) and a surgical visualization system (e.g., a digital surgical microscope (DSM) 1044). Also or alternatively, the surgical navigation system and the surgical visualization system may be integrated. In some embodiments, the surgical navigation and visualization system(s) 1040 may comprise or include, and/or may be communicatively linked to, the computing device 1002. Also or alternatively, the computing device 1002 may be a standalone device in the vicinity of the surgical navigation and visualization system(s) 1040 (e.g., within a cart associated with the surgical navigation and visualization system(s) 1040). The surgical navigation and visualization system(s) 1040 may include one or more robotic arm controls 1042 (e.g., actuators) to control one or more robotic arms to cause movement of the DSM 1044, e.g., in response to commands 1030 received from the computing device 1002. The movement may realign the field of view of a DSM camera 1047 of the DSM 1044, e.g., in order to track a tool of interest. The DSM 1044 may include the DSM camera 1047 and controls for one or more camera settings (e.g., settings 1046). In some embodiments, one or more settings 1046 of the DSM camera 1047 may be adjusted based on the computing device 1002 detecting user intent, e.g., by relying on a neural network model to detect gestures used by the surgeon's hand and/or positions of the tool of interest. In some aspects, the localizer 1041 may report pose information for tools in the field of view of the DSM camera 1047. The DSM camera 1047 may comprise one or more image sensors 1048 that may capture one or more images or a video stream of a field of view, in real-time, and generate image data (e.g., image data 1020). The image data 1020 may be sent to and/or received by the computing device 1002. The field of view of the DSM camera 1047 may capture at least a portion of a surgical site 1050, as shown in FIG. 10.

The surgical site 1050 may comprise a patient anatomy 1058 to which a surgeon may be performing a surgical procedure or operation via a tool 1052. The distal end 1054 of the tool 1052 may be identified and detected by the computing device 1002 using image data 1020 based on methods discussed herein. The computing device 1002 may cause the DSM camera 1047 to track the detected distal end 1054 of the tool 1052, e.g., by causing the robotic arm controls 1042 to situate the DSM camera 1047 to bring the distal end 1054 at the center of the field of view. Also or alternatively, the computing device may track a location 1056 that is at a predetermined distance from the distal end 1054 of the tool 1052. As the surgeon holds and/or maneuvers the tool 1052 during the surgical procedure, the surgeon's hand 1060 may be a part of the field of view. In some embodiments, the surgeon's hand 1060 and/or the tool 1052 may be used to form gestures to signal a user intent. Image data capturing such gestures may be inputted into one or more neural network models 1010 at the computing device 1002 to detect the user intent. One or more settings 1046 of the DSM 1044 may be adjusted based on the user intent.

The computing device 1002 may comprise a processor 1004 and memory 1006. The processor 1004 may comprise, for example, a central processing unit (CPU) processor; a graphics processing unit (GPU); a specialized GPU tailored to perform inference or machine learning from largescale training data (e.g., a “tensor core” present in NVIDIA'S VOLTA and TURING architectures), a dedicated inference device (e.g., INTEL® NEURAL COMPUTE STICK 2), or a cloud-based solutions on the Internet. The memory 1006 may comprise any type of long term, short term, volatile, nonvolatile, or other memory and is not to be limited to any particular type of memory or number of memories, or type of media upon which memory is stored. The memory may store computer executable instructions 1008 that, when executed by the processor 1004, can cause the processor 1004, the computing device 1002, and/or the system 1000 to perform one or more methods discussed herein.

For example, the computing device 1002 may receive, in real-time, image data 1020 of a surgical video stream captured by the DSM camera 1047. The surgical video stream may show a portion of the surgical site 1050, and that portion may show a tool of interest (e.g., tool 1052). The computing device 1002 may apply the image data 1020 to a trained neural network model stored in memory 1006 (e.g., as one or more neural network models 1010) to determine a location for a bounding box around the tool of interest 1052. The computing device 1002 may generate an augmented image data 1012 comprising a bounding box around the tool of interest. In some aspects, the augmented image data 1012 may be temporarily stored in the memory 1006. The computing device may apply the augmented image data 1012 to another trained neural network model (second trained neural network model) to determine a distal end point of the tool of interest 1014. The second trained neural network model may also be trained, stored, and/or retrieved from memory 1006. Furthermore, the computing device 1002 may cause, in real-time, the DSM camera 1047 to track the distal end 1054 of the tool of interest 1052 (and/or an identified point 956 associated with the distal end 954). For example, the computing device 1002 may generate commands 1030 causing the robotic arm controls 1042 of the surgical navigation and visualization system(s) 1040 to adjust the DSM camera 1047 to follow the distal end 1054 and/or the identified point 1056.

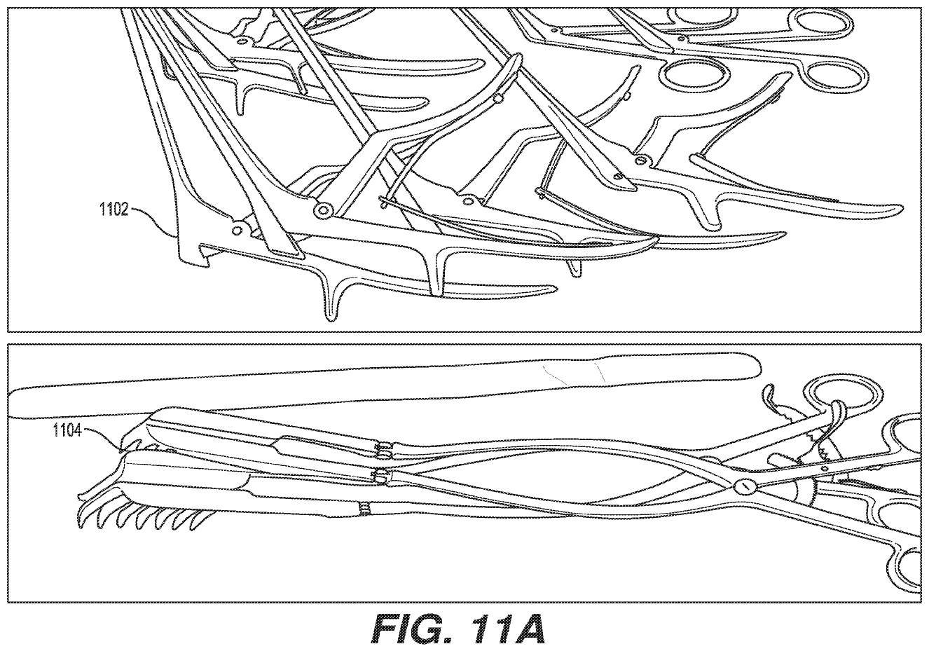

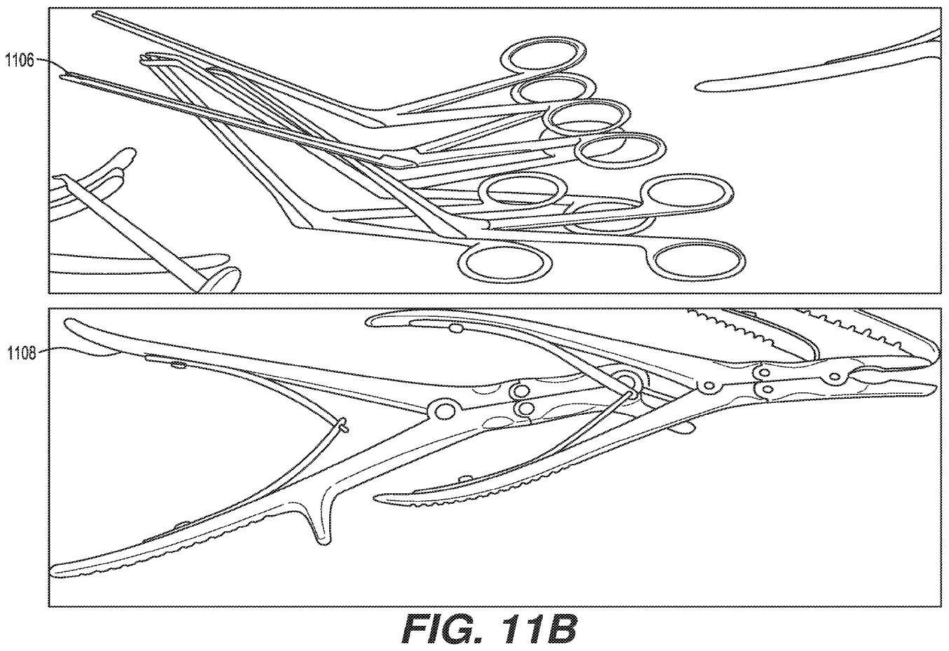

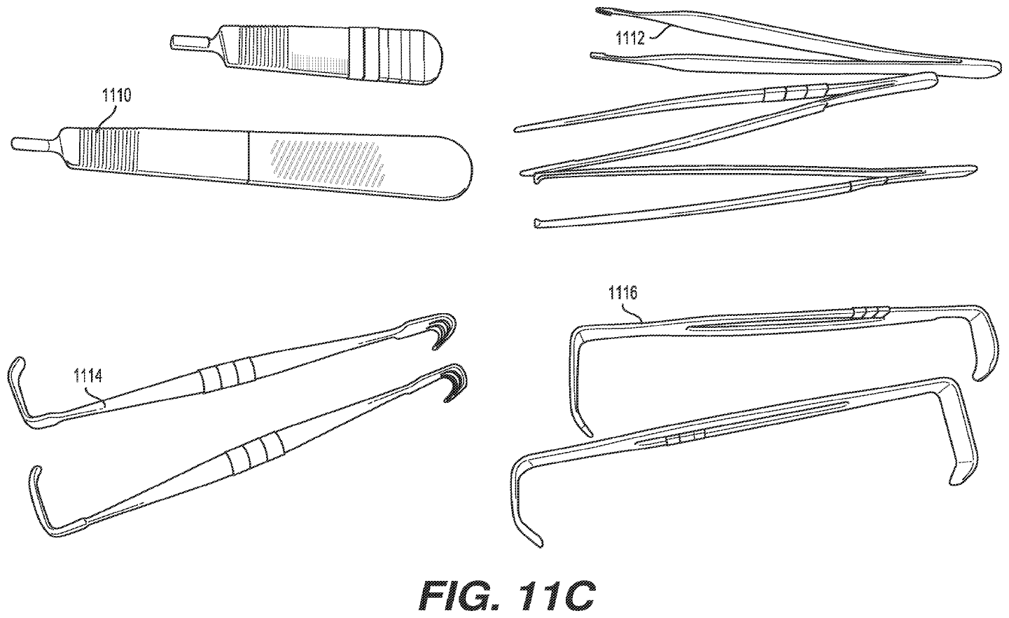

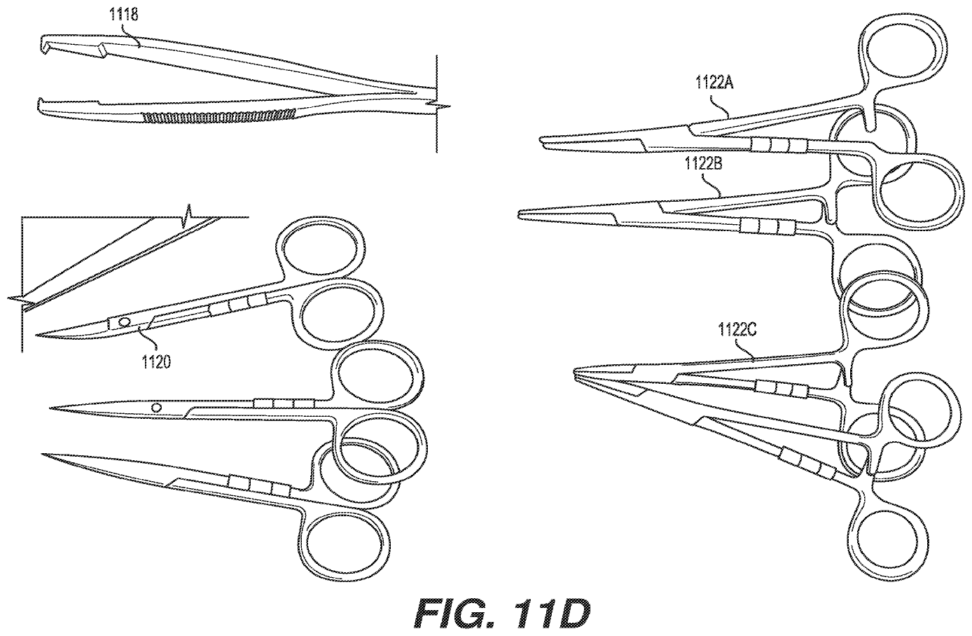

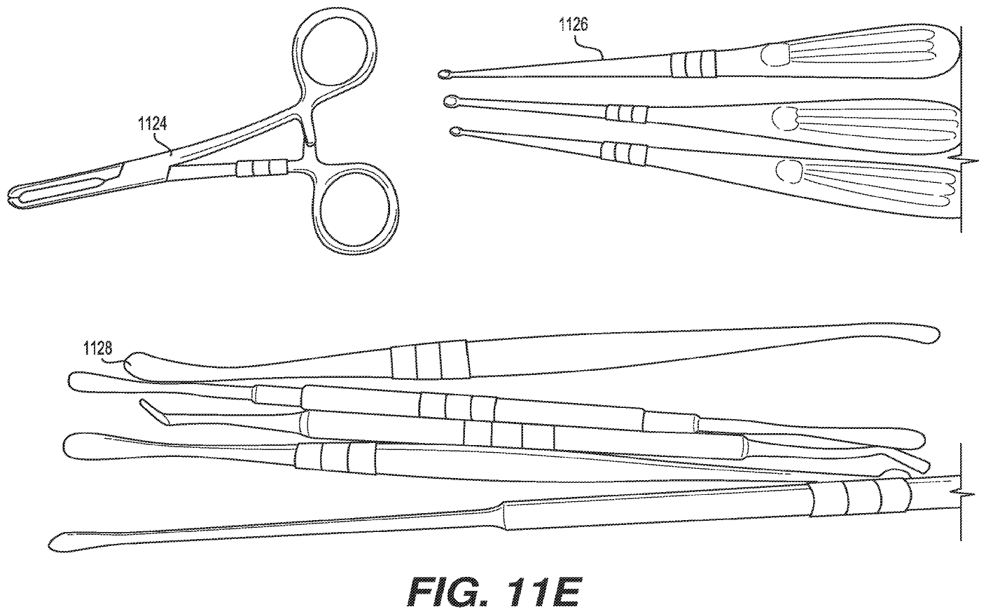

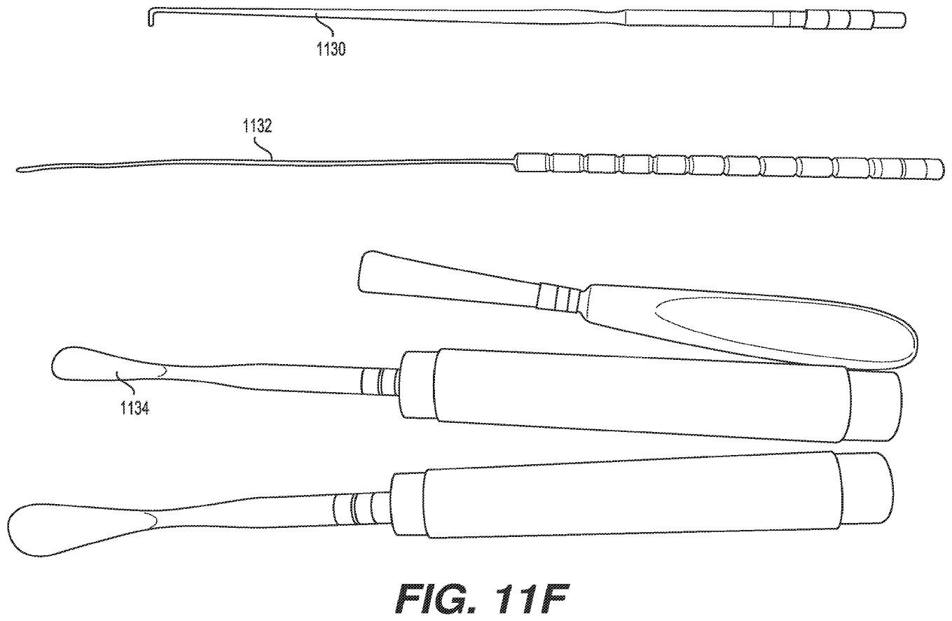

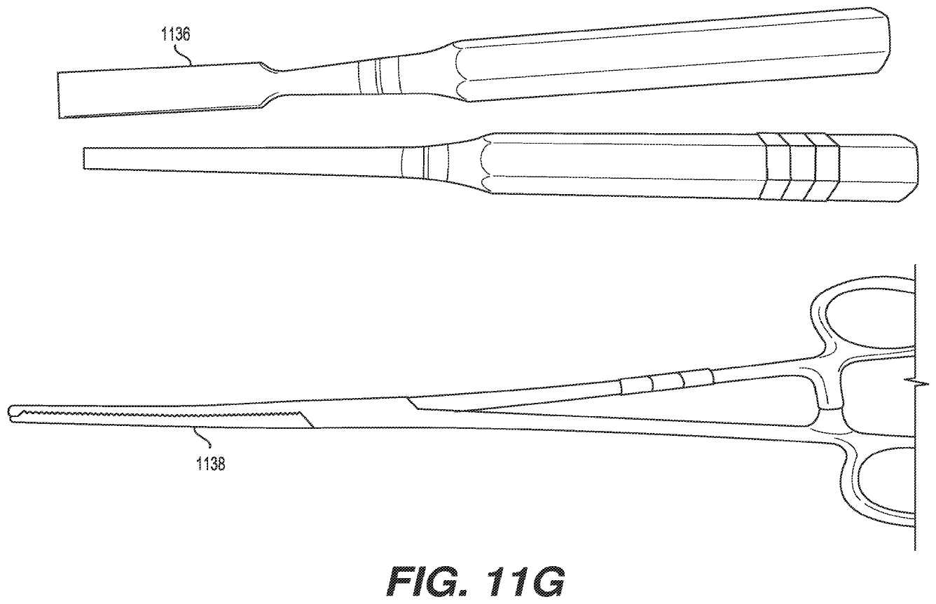

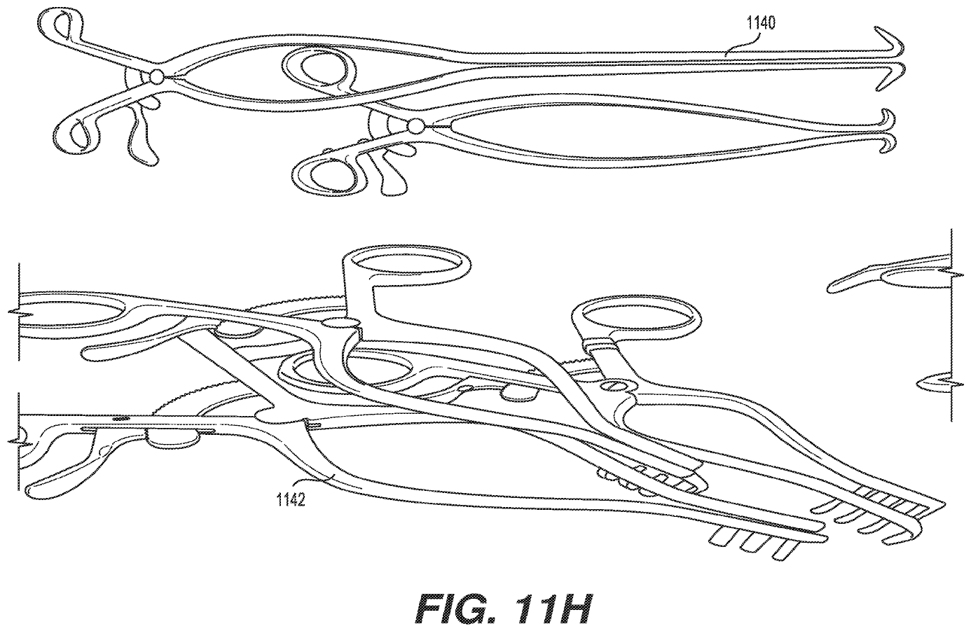

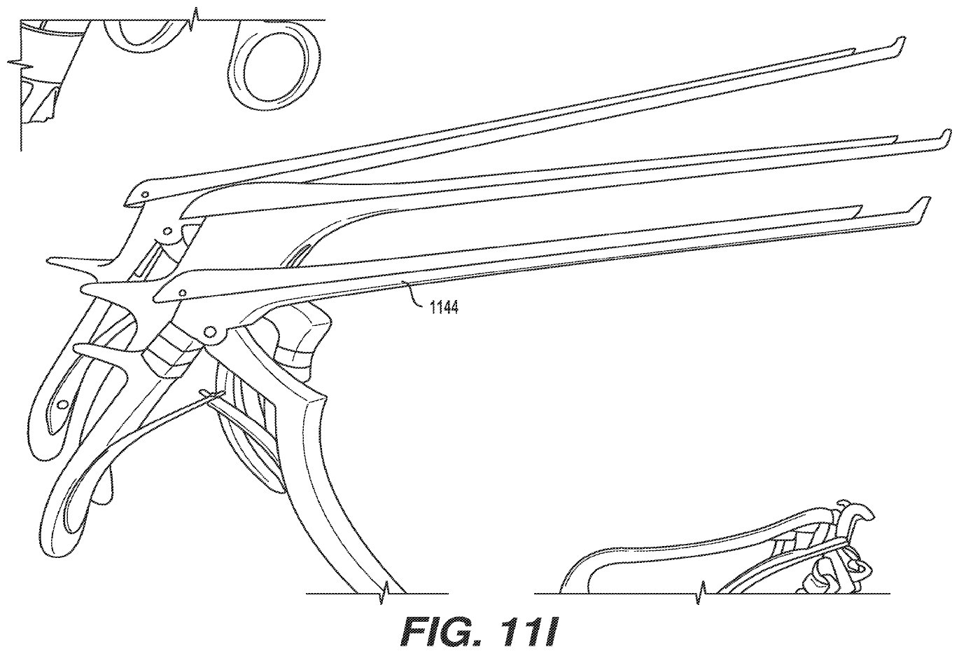

FIGS. 11A-11I show illustrations of example tools of interest, according to example embodiments of the present disclosure. For example, FIG. 11A shows a Kerrison rongeur 1102 (top) and a spinal self-retaining retractor 1104 (bottom). FIG. 11B shows a pituitary rongeur 1106 (top) and a lexcel rongeur 1108 (bottom). FIG. 11C shows a bladeless knife handle 1110 (top left), tissue forceps 1112 (top right), a Senn handheld retractor 1114 (bottom left), handheld retractor 1116 (bottom right). FIG. 11D shows a bonnet 1118 (top left), iris scissors 1122 (bottom left), and hemostats (e.g., a straight hemostat 1122A, a curved hemostat 1122B, a mosquito hemostat 1122C) (right). FIG. 11E shows a Babcock forcep 1124 (top left), curets 1126 (e.g., up biting, down biting, regular, ring, etc.) (top right), and Pennfield freers 1128 (bottom). FIG. 11F shows a nerve hook probe 1130 (top), a ball tip probe 1132 (second from top), and posterior cobb elevator 1134 (bottom). FIG. 11G shows an osteotome 1136 (top) and an Oschner forcep 1138 (bottom). FIG. 11H shows Gelpi retractors 1140 (top) and Weitlaner retractors 1142 (bottom). FIG. 11I shows Kerrison Rongeurs 1144. As previously discussed, the first neural network model may be trained to differentiate and determine a bounding box around any one or more of the tools of interest. Furthermore, the second neural network model may be trained to identify a distal end of the tool of interest. It is to be appreciated that the tools shown in FIGS. 11A-11I are not exhaustive of the tools of interest employed in the systems and methods presented herein. Any tool may be deemed, added, or deleted from the tools of interest, as appropriate.

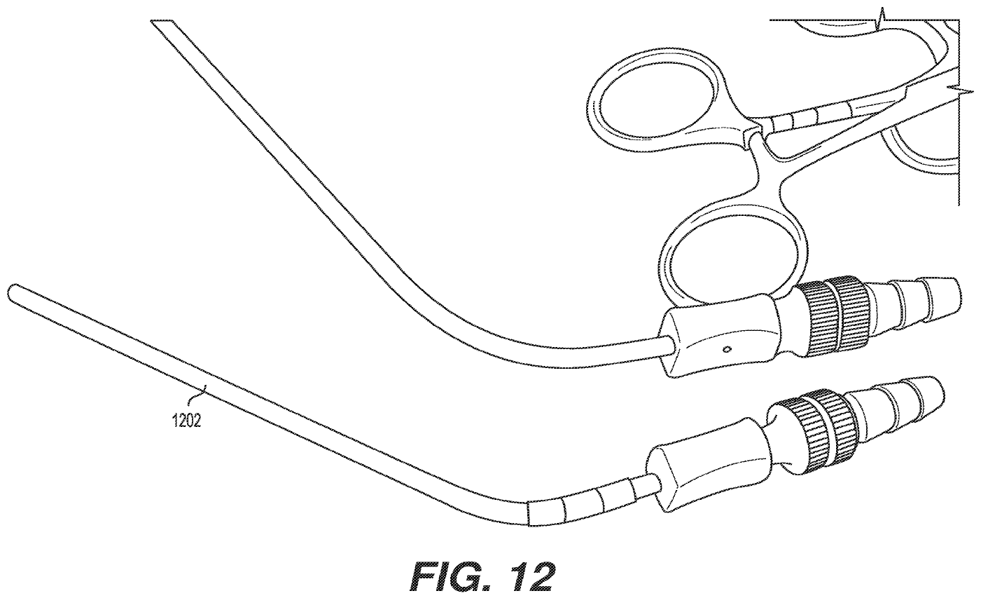

FIG. 12 shows an illustration of an example non-relevant tool, according to example embodiments of the present disclosure. Specifically, FIG. 12 shows a Frazier suction tool 1202. As previously discussed, the first neural network model may be trained to differentiate between tools of interest (e.g., tools shown in FIGS. 11A-11I) and non-relevant tools (e.g., Frazier suction tool 1202). In some embodiments output vectors indicating location and other details (e.g., aspect ratios) for bounding boxes around tools may also indicate (e.g., via a value) whether a tool is of a tool of interest or a non-relevant tool. In some aspects, all suction devices and respirators may be deemed as non-relevant tools.

It will be appreciated that all of the disclosed methods and procedures described herein may be implemented using one or more computer programs or components. These components may be provided as a series of computer instructions on any conventional computer readable medium or machine readable medium, including volatile or non-volatile memory, such as RAM, ROM, flash memory, magnetic or optical disks, optical memory, or other storage media. The instructions may be provided as software or firmware, and/or may be implemented in whole or in part in hardware components such as ASICs, FPGAs, DSPs or any other similar devices. The instructions may be configured to be executed by one or more processors, which when executing the series of computer instructions, performs or facilitates the performance of all or part of the disclosed methods and procedures.

It should be understood that various changes and modifications to the example embodiments described herein will be apparent to those skilled in the art. Such changes and modifications may be made without departing from the spirit and scope of the present subject matter and without diminishing its intended advantages. It is therefore intended that such changes and modifications be covered by the appended claims. To the extent that any of these aspects are mutually exclusive, it should be understood that such mutual exclusivity shall not limit in any way the combination of such aspects with any other aspect whether or not such aspect is explicitly recited. Any of these aspects may be claimed, without limitation, as a system, method, apparatus, device, medium, etc.

What is claimed is:

1. A method of tracking a surgical tool in real-time via a digital surgical microscope (DSM), the method comprising:
receiving, in real-time, by a computing device having a processor, image data of a surgical video stream captured by a camera of the DSM, wherein the surgical video stream shows a tool of interest;
applying the image data to a first trained neural network model to determine a location for a bounding box around the tool of interest;
generating augmented image data comprising a bounding box around the tool of interest;
applying the augmented image data to a second trained neural network model to determine a distal end point of the tool of interest; and
causing, in real-time by the computing device, the DSM camera to track the distal end of the tool of interest.

2. The method of claim 1, further comprising:
identifying, by the computing device, based on a previously received image data of the surgical video stream, a displacement of a feature in the surgical video stream beyond a threshold distance,
wherein the applying the image data, the generating the augmented image data, the applying the augmented image data, and the causing the DSM camera to track the distal end of the tool of interest is responsive to the identified displacement.

3. The method of claim 2, wherein causing the DSM camera to track the distal end of the tool of interest comprises:
adjusting, by the computing device, a field of view of the DSM camera such that a focus point associated with the distal end of the tool of interest is at the center of the field of view, wherein the focus point is at a predetermined distance from the distal end of the tool of interest in a direction towards the displacement.

4. The method of claim 1, wherein applying the image data to the first trained neural network model comprises:
generating a first input feature vector based on the image data; and
applying the first input feature vector to the first trained neural network model to generate a first output feature vector, wherein the first output feature vector indicates the location for the bounding box around the tool of interest.

5. The method of claim 1, wherein applying the augmented image data to the second trained neural network model comprises:
generating a second input feature vector based on the augmented image data; and
applying the second input feature vector to the second trained neural network model to generate a second output feature vector, wherein the second output feature vector indicates the location for the distal end of the tool of interest.

6. The method of claim 1, further comprising, prior to applying the image data to the first trained neural network model:
receiving a plurality of reference image data of a reference surgical video, wherein each reference image data represents a respective image frame showing a plurality of tools including the tool of interest;
generating, for each of the plurality of reference image data,
a reference input feature vector indicating relevant features of the reference image data, and
a reference output feature vector indicating a location for a bounding box around the tool of interest; and
training, by associating each reference input feature vector with its respective reference output feature vector, a neural network to form the first trained neural network model.

7. The method of claim 1, further comprising, prior to applying the augmented image data to the second trained neural network model:
receiving a plurality of reference image data representing a plurality of respective reference images showing a plurality of respective reference surgical tools;
generating, for each of the plurality of reference image data,
a reference input feature vector indicating relevant features of the reference image data, and
a reference output feature vector indicating a location for a distal end of a reference surgical tool; and
training, by associating each reference input feature vector with its respective reference output feature vector, a neural network to form the second trained neural network model.

8. The method of claim 1, further comprising:
applying, by the computing device, one or more image data of the surgical video stream to a third neural network model to detect a user intent; and
altering, based on the user intent, one or more settings of the DSM camera.

9. A system for tracking a surgical tool in real-time via a digital surgical microscope (DSM), the system comprising:
a DSM comprising a DSM camera;
a processor; and
a memory device storing computer-executable instructions that, when executed by the processor, causes the processor to:
receive, in real-time, image data of a surgical video stream captured by the DSM camera, wherein the surgical video stream shows a tool of interest;
apply the image data to a first trained neural network model to determine a location for a bounding box around the tool of interest;
generate an augmented image data comprising a bounding box around the tool of interest;
apply the augmented image data to a second trained neural network model to determine a distal end point of the tool of interest; and
cause, in real-time, the DSM camera to track the distal end of the tool of interest.

10. The system of claim 9, wherein the instructions, when executed, further causes the processor to:
identify, based on a previously received image data of the surgical video stream, a displacement of a feature in the surgical video stream beyond a threshold distance,
wherein the applying the image data, the generating the augmented image data, the applying the augmented image data, and the causing the DSM camera to track the distal end of the tool of interest is responsive to the identified displacement.

11. The system of claim 10, wherein the instructions, when executed, causes the processor to cause the DSM camera to track the distal end of the tool of interest by:
adjusting a field of view of the DSM camera such that a focus point associated with the distal end of the tool of interest is at the center of the field of view, wherein the focus point is at a predetermined distance from the distal end of the tool of interest in a direction towards the displacement.

12. The system of claim 9, wherein the instructions, when executed, causes the processor to apply the image data to the first trained neural network model by:
generating a first input feature vector based on the image data; and
applying the first input feature vector to the first trained neural network model to generate a first output feature vector, wherein the first output feature vector indicates the location for the bounding box around the tool of interest.

13. The system of claim 9, wherein the instructions, when executed, causes the processor to apply the augmented image data to the second trained neural network model by:
generating a second input feature vector based on the augmented image data; and
applying the second input feature vector to the second trained neural network model to generate a second output feature vector, wherein the second output feature vector indicates the location for the distal end of the tool of interest.

14. The system of claim 9, wherein the instructions, when executed, further causes the processor to, prior to applying the image data to the first trained neural network model:
receive a plurality of reference image data of a reference surgical video, wherein each reference image data represents a respective image frame showing a plurality of tools including the tool of interest;
generate, for each of the plurality of reference image data,
a reference input feature vector indicating relevant features of the reference image data, and
a reference output feature vector indicating a location for a bounding box around the tool of interest; and
train, by associating each reference input feature vector with its respective reference output feature vector, a neural network to form the first trained neural network model.

15. The system of claim 9, wherein the instructions, when executed, further causes the processor to, prior to applying the augmented image data to the second trained neural network model:
receive a plurality of reference image data representing a plurality of respective reference images showing a plurality of respective reference surgical tools;
generate, for each of the plurality of reference image data,
a reference input feature vector indicating relevant features of the reference image data, and
a reference output feature vector indicating a location for a distal end of a reference surgical tool; and
train, by associating each reference input feature vector with its respective reference output feature vector, a neural network to form the second trained neural network model.

16. The system of claim 9, wherein the instructions, when executed, further causes the processor to:
apply one or more image data of the surgical video stream to a third neural network model to detect a user intent; and
alter, based on the user intent, one or more settings of the DSM camera.

17. The system of claim 9, wherein the first trained neural network model is trained to differentiate between the tool of interest and one or more suction tools, and wherein the tool of interest is not any of the one or more suction tools.

18. A method of tracking a tool in real time via a digital surgical microscope (DSM), the method comprising:
receiving, from one or more image sensors associated with the digital surgical microscope, by a computing device having a processor, image data of a first segment of a medical procedure, wherein the one or more image sensors are focused towards a first position of a surgical area of the medical procedure;
applying, by the computing device, at least one of a computer vision tracking algorithm or a neural network to the image data to detect a tool;
identifying, by the computing device, and based on the location of the detected tool, a central region of the medical procedure, wherein the threshold distance is from the location of the detected tool to the edge of the central region
tracking, by the computing device, movement of the tool through one or more subsequent segments after the first segment; and
after detecting a displacement of the tool beyond a threshold distance, refocusing the image sensors towards a second position of the surgical area of the medical procedure.

19. The method of claim 18, further comprising, prior to applying the neural network to the image data to detect the tool,
receiving, by the computing device, a plurality of reference image data showing a plurality of reference tool markings;
generating, by the computing device, a plurality of feature vectors corresponding to the plurality of reference image data showing the plurality of reference tool markings, wherein each feature vector is generated via a convolution of the respective reference image data;
associating, by the computing device, each of the plurality of feature vectors with their respective tool marking; and
training, by the computing device, the neural network, wherein the detecting the tool is based on a tool marking of the tool.

20. The method of claim 18, further comprising:
applying, by the computing device, a second neural network to the image data of the one or more subsequent segments, to detect a user intent; and
altering, based on the user intent, one or more settings of the image sensors.
